SEQUENCE LISTING (40 sequences)

SEQ ID NO: 1 - protein, 36 aa, Homo sapiens
  Amidation (36)..(36)
YPSKPDNPGEDAPAEDMARYYSALRHYINLITRQRY

SEQ ID NO: 2 - protein, 36 aa, Homo sapiens
  Amidation (36)..(36)
YPIKPEAPGEDASPEELNRYYASLRHYLNLVTRQRY

SEQ ID NO: 3 - protein, 36 aa, Homo sapiens
  Amidation (36)..(36)
APLEPVYPGDNATPEQMAQYAADLRRYINMLTRPRY

SEQ ID NO: 4 - protein, 36 aa, Artificial
  Modified PP
  Amidation (36)..(36)
APLEPVYPGDNATPEQMAQYAADLRRYINMLTRQRY

SEQ ID NO: 5 - protein, 36 aa, Artificial
  Modified PP
  Amidation (36)..(36)
APLEPVYPGDNATPEQMAQYAADLRRYINMITRQRY

SEQ ID NO: 6 - protein, 36 aa, Artificial
  Modified PP
  Amidation (36)..(36)
APLEPVYPGDNATPEQMAQYAADLRRYINMVTRQRY

SEQ ID NO: 7 - protein, 36 aa, Artificial
  Modified PP
  Amidation (36)..(36)
APLEPVYPGDNATPEQMAQYAADLRRYINLLTRQRY

SEQ ID NO: 8 - skipped

SEQ ID NO: 9 - protein, 36 aa, Artificial
  Modified PP
  Amidation (36)..(36)
APLEPVYPGDNATPEQMAQYAADLRRYLNMLTRQRY

SEQ ID NO: 10 - protein, 36 aa, Artificial
  Modified PP
  Amidaition (36)..(36)
APLEPVYPGDNATPEQMAQYAADLRHYINMLTRQRY

SEQ ID NO: 11 - protein, 36 aa, Artificial
  Modified PP
  Amidaition (36)..(36)
APIEPVYPGDNATPEQMAQYAADLRRYINMLTRQRY

SEQ ID NO: 12 - protein, 36 aa, Artificial
  Modified PYY
  misc_feature (30)..(30): Xaa can be any naturally occurring amino acid
  Amidation (36)..(36)
  Amidation (36)..(36): X = Met or Nle
APIEPEAPGEDASPEELNRYYASLRRYLNXVTRQRY

SEQ ID NO: 13 - protein, 36 aa, Artificial
  Modified NPY
  misc_feature (30)..(30): Xaa can be any naturally occurring amino acid
  Amidation (36)..(36): X = Met or Nle
APSEPDNPGEDAPAEDMARYYSALRRYINXITRQRY

SEQ ID NO: 14 - protein, 36 aa, Artificial
  Modified PYY
  Amidation (36)..(36)
APIEPEAPGEDASPEELNRYYASLRHYLNLVTRQRY

SEQ ID NO: 15 - protein, 36 aa, Artificial
  Modified NPY
  Amidation (36)..(36)
APSEPDNPGEDAPAEDMARYYSALRHYINLITRQRY

SEQ ID NO: 16 - protein, 36 aa, Artificial
  Modified PYY
  misc_feature (30)..(30): Xaa can be any naturally occurring amino acid
  Amidation (36)..(36): X = Met or Nle
YPIKPEAPGEDASPEELNRYYASLRRYLNXVTRQRY

SEQ ID NO: 17 - protein, 36 aa, Artificial
  Modified NPY
  misc_feature (30)..(30): Xaa can be any naturally occurring amino acid
  Amidation (36)..(36)
  Amidation (36)..(36): X = Met or Nle
YPSKPDNPGEDAPAEDMARYYSALRRYINXITRQRY

SEQ ID NO: 18 - protein, 36 aa, Artificial
  Modified PYY
  misc_feature (30)..(30): Xaa can be any naturally occurring amino acid
  Amidation (36)..(36): X = Met or Nle
YPIEPEAPGEDASPEELNRYYASLRHYLNXVTRQRY

SEQ ID NO: 19 - protein, 36 aa, Artificial
  Modified NPY
  misc_feature (30)..(30): Xaa can be any naturally occurring amino acid
  Amidation (36)..(36)
  Amidation (36)..(36): X = Met or Nle
YPSEPDNPGEDAPAEDMARYYSALRHYINXITRQRY

SEQ ID NO: 20 - protein, 36 aa, Artificial
  Modified PYY
  Amidation (36)..(36)
YPIEPEAPGEDASPEELNRYYASLRRYLNLVTRQRY

SEQ ID NO: 21 - protein, 36 aa, Artificial
  Modified NPY
  Amidation (36)..(36)
YPSEPDNPGEDAPAEDMARYYSALRRYINLITRQRY

SEQ ID NO: 22-23 - skipped (2 entries)

SEQ ID NO: 24 - protein, 36 aa, Artificial
  Modified PP
  Amidation (36)..(36)
APLEPVYPGDNATPEQMKQYAADLRRYINMLTRQRY

SEQ ID NO: 25 - protein, 36 aa, Artificial
  Modified PYY
  misc_feature (30)..(30): Xaa can be any naturally occurring amino acid
  Amidation (36)..(36): X = Met or Nle
APIEPEAPGEKASPEELNRYYASLRRYLNXVTRQRY

SEQ ID NO: 26 - protein, 60 aa, Artificial
  Modified PP
  Amidation (60)..(60)
ARRRAARAARRRAARAARRRAARAAPLEPVYPGDNATPEQMAQYAADLRRYINMLTRQRY

SEQ ID NO: 27 - protein, 36 aa, Artificial
  Modified PYY
  misc_feature (30)..(30): Xaa can be any naturally occurring amino acid
  Amidation (36)..(36)
APIEPEAPGEDASPEELKRYYASLRRYLNXVTRQRY

SEQ ID NO: 28 - protein, 11 aa, Artificial
  Truncated, modified PP
  Amidation (11)..(11)
RYKNMLERPRY

SEQ ID NO: 29 - protein, 42 aa, Artificial
  Modified PP
  Amidation (42)..(42)
KKKKKKAPLEPVYPGDNATPEQMAQYAADLRRYINMLTRQRY

SEQ ID NO: 30-40 - skipped (11 entries)

(19) United States
(12) Patent Application Publication
(10) Pub. No.: US 2008/0261871 A1
(43) Pub. Date: Oct. 23, 2008

(54) Y2/Y4 Selective Receptor Agonists for Therapeutic Interventions

(75) Inventor: Thue Schwartz, Frederiksberg (DK)

Correspondence Address: BANNER & WITCOFF, LTD., 1100 13th STREET, N.W., SUITE 1200, WASHINGTON, DC, 20005-4051, US

(73) Assignee: 7TM PHARMA A/S, Hoersholm (DK)

(21) Appl. No.: 10/592,688

(22) PCT Filed: Mar. 17, 2005
(86) PCT No.: PCT/EP2005/002982

§ 371 Date: Jul. 9, 2007

Related U.S. Application Data
(60) Provisional application No. 60/558,932, filed on Apr. 1, 2004.
(60) Provisional application No. 60/585,964, filed on Jul. 6, 2004.

(30) Foreign Application Priority Data
Mar. 17, 2004 (DK) PA 2004 00436
Feb. 14, 2005 (GB) 0503111.7

(51) Int. Cl. A61K 38/16; C07K 14/00; A61P 3/00; A61P 9/00; A61P 35/00

(52) U.S. Cl. 514/12; 530/324

(57) ABSTRACT

Y receptor agonists which are selective for Y2 and Y4 receptors over the Y1 receptor are useful for treatment of, for example obesity, are (a) PP-fold peptides or PP-fold peptides mimics which have (i) a C-terminal Y receptor-recognition amino acid sequence represented by —X-Thr-Arg-X³-Arg-Tyr-C(=0)NR¹R² wherein R¹ and R¹ are independently hydrogen or C₁-C₆ alkyl X is Val, Ile, Leu or Ala, and X³ is Gln or Asn, or a conservatively substituted variant thereof in which Thr is replaced by His or Asn and/or Tyr is replaced by Trp or Phe; and/or Arg is replaced by Lys, and (ii) an N-terminal Y receptor-recognition amino acid sequence represented by H₂N—X¹-Pro-X²—(Glu or Asp)—wherein X¹ is not present or is amino acid residue, and X² is Leu or Ser or conservative substitutions of Leu or Ser, or (b) the said comprise a C-terminal Y receptor-recognition amino acid sequence as defined in (i) above, said Y receptor-recognition sequence being fused to an amphiphilic amino acid sequence domain comprising at least one alpha helical turn adjacent the N-terminus of the said hexapeptide sequence, said turn being constrained in a helical configuration by a covalent intramolecular link, and optionally an N-terminal sequence which commences with a Y receptor-recognition amino acid sequence as defined in (ii) above.

### FIELD OF THE INVENTION

The invention relates to peptide or peptidic compounds that act as selective agonists of the Y2 and Y4 relative to the Y1 receptors, and to their use in treatment of conditions responsive to the activation of Y2 and/or Y4 receptors, for example treatment of obesity and overweight, and conditions in which these are considered contributory factors and for controlling/decreasing GI-tract secretion

### BACKGROUND TO THE INVENTION

The PP-fold family of peptides —NPY (Neuropeptide Y) (human sequence—SEQ ID. No:1), PYY (Peptide YY) (human sequence—SEQ ID. No:2), and PP (Pancreatic Polypeptide) (human sequence—SEQ ID. No:3), are naturally secreted homologous, 36 amino acid, C-terminally amidated peptides, which are characterized by a common three-dimensional, structure—the PP-fold—which is surprisingly stable even in dilute aqueous solution and is important for the receptor recognition of the peptides.

Initially the X-ray structure of avian PP was characterized in great detail through X-ray crystallographic analysis down to a resolution of 0.98 Å and the unique structure obtained its name from this peptide (Blundell et al. 1981 Proc. Natl. Acad. Sci. USA 78: 4175-79; Glover et al. 1984, Eur. J. Biochem. 142: 379-85). Subsequently, the PP-fold structure of other members of the family have been analysed through especially NMR spectroscopic analysis. Both X-ray and NMR analysis are obviously performed in very concentrated or solid conditions; however, detailed circular dichroism analysis suggests that NPY and PP even in aqueous solution adopt the PP-fold structure, which is unusual for such a small peptide (Fuhlendorff et al. 1990 J. Biol. Chem. 265: 11706-12). Importantly, analysis of the proteolytic stability of the peptides and fragments and analogs of these strongly indicate that for example the full length PP1-36 even in dilute aqueous solution is held in a folded configuration which protects it from degradation by certain enzymes which readily and rapidly degrade analogs which cannot adopt the PP-fold structure due to minor substitutions (Schwartz et al., 1990 Annals NY Acad. Sci. 611: 35-47).

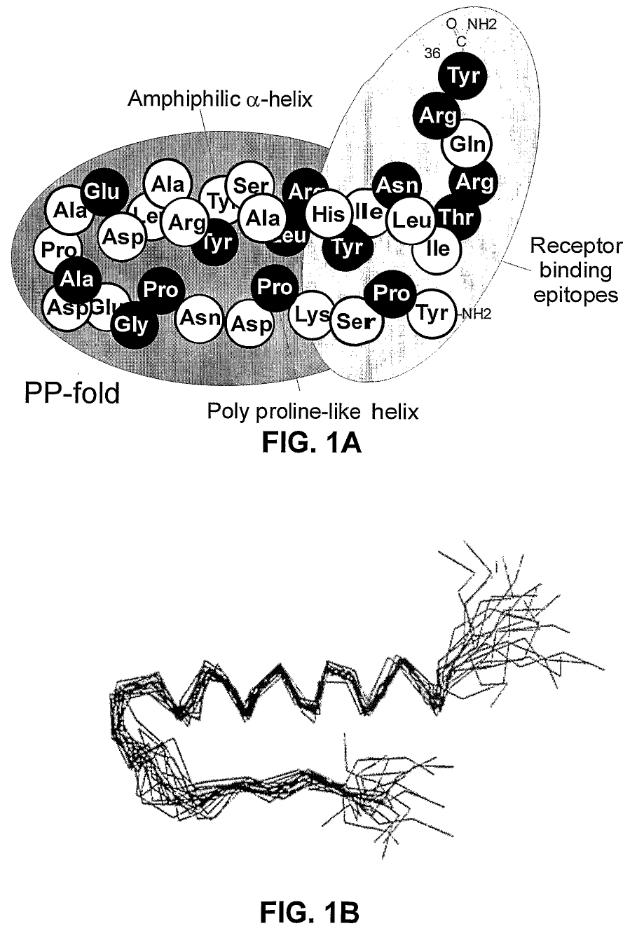

The PP-fold structure common to NPY, PYY and PP consists of 1) an N-terminal polyproline-like helix (corresponding to residues 1 through 8 with Pro2, Pro5, and Pro8) followed by 2) a type I beta-turn region (corresponding to residues 9 through 12) followed by 3) an amphiphilic alpha-helix (residues 13-30) which lies anti-parallel to the polyproline helix with an angle of about 152 degrees between the helical axes, and 4) a C-terminal hexapeptide (residues 31-36). The folded structure is stabilized through hydrophobic interactions between side chains of the amphiphilic alpha-helix which are closely interdigitating with the three hydrophobic proline residues (Schwartz et al 1990). Besides key residues in the receptor recognizing C-terminal hexapeptide it is the core hydrophobic residues, which stabilize the PP-fold structure, which are conserved across the family of PP-fold peptides. FIG. 1A depicts the NPY sequence, with residues which are conserved amongst NPY, PYY and PP shown as white text on dark background. FIG. 1A also illustrates the elements of the PP-fold structure described above. The C-terminal hexapeptide, which is important for receptor recognition is believed to be unstructured, but the PP fold provides a stable scaffold, which presents the C-terminal hexapeptide to the receptors (illustrated in FIG. 1B), which to variable degree are dependent or independent also upon parts of the N-terminus of the peptides. NMR spectroscopic analysis has demonstrated that the far C- and the N-terminal parts of for example NPY are rather mobile, meaning that the PP-fold is constantly in danger of being “unzipped” from the free terminal end.

NPY is a very wide-spread neuropeptide with multiple actions in various parts of both the central and peripheral nervous system acting through a number of different receptor subtypes in man: Y1, Y2, Y4 and Y5. The main NPY receptors are the Y1 receptor, which generally is the post-synaptic receptor conveying the “action” of the NPY neurones and the Y2 receptor which generally is a pre-synaptic, inhibitory receptor. This is also the case in the hypothalamus, where NPY neurones—which also express the melanocortin receptor antagonist/inverse agonist AgRP (agouti related peptide)—act as the primary “sensory” neurones in the stimulatory branch of the arcuate nucleus. Thus, in this the “sensor nucleus” for the control of appetite and energy expenditure, the NPY/AgRP neurones together with the inhibitory POMC/CART neurones monitor the hormonal and nutritional status of the body as these neurones are the target for both the long-term regulators such as leptin and insulin and short term regulators such as ghrelin and PYY (see below). The stimulatory NPY/AgRP neurones project for example to the paraventricular nucleus—also of the hypothalamus—where its postsynaptic target receptors are believed to be Y1 and Y5 receptors. NPY is the most potent compound known in respect of increasing food intake, as rodents upon intracerebroventricular (ICV) injection of NPY will eat until they literally burst. AgRP from the NPY/AgRP neurones acts as an antagonist mainly on melanocortin receptors type 4 (MC-4) and block the action of POMC derived peptides—mainly aMSH—on this receptor. Since the MC4 receptor signal acts as an inhibitor of food intake, the action of AgRP is—just like the NPY action—a stimulatory signal for food intake (i.e. an inhibition of an inhibition). On the NPY/AGRP neurons are found inhibitory—pre-synaptic—Y2 receptors, which are the target both of locally released NPY as well as a target for the gut hormone PYY —another PP-fold peptide.

PYY is released during a meal—in proportion to the calorie content of the meal—from entero-endocrine cells in the distal small intestine and the colon, to act both in the periphery on GI-tract functions and centrally as a satiety signal. Peripherally, PYY is believed to function as an inhibitor—an “illeal break”—on for example upper GI-tract motility, gastric acid and exocrine pancreatic secretion. Centrally, PYY is believed to act mainly on the presynaptic, inhibitory Y2 receptors on the NPY/AgRP neurones in the arcuate nucleus, which it is believed to get access to from the blood (Batterham et al. 2002 Nature 418: 650-4). The peptide is released as PYY1-36, but a fraction—approximately 50%—circulates as PYY3-36 which is a product of degradation by dipeptidylpeptidase-IV an enzyme which removes a dipeptide from the N-terminus of a peptide provided that a Pro or Ala is found in position two as in all three PP-fold peptides —PP, PYY and NPY (Eberlein et al. 1989 Peptides 10: 797-803). Thus PYY in the circulation is a mixture of PYY1-36, which acts on both Y1 and Y2 receptors (as well as Y4 and Y5 with various affinities), and PYY3-36—which has lower affinities for the Y1, Y4 and Y5 receptors than for the Y2 receptor.

PP is a hormone, which is released from endocrine cells in the pancreatic islets, almost exclusively governed by vagal cholinergic stimuli elicited by especially food intake (Schwartz 1983 Gastroenterology 85:1411-25). PP has various effects on the gastrointestinal tract, but these are generally not observed in isolated cells and organs, and appear to be dependent on an intact vagal nerve supply (Schwartz 1983 Gastroenterology 85:1411-25). In accordance with this, the PP receptors, which are called Y4 receptors, are located mainly in area postrema in the brain stem with a strong expression in vagal motor neurones—activation of which results in the peripheral effects of PP—and in the nucleus tractus solitarirus (NTS)—activation of which results in the effects of PP as a satiety hormone (Whitecomb et al. 1990 Am. J. Physiol. 259: G687-91, Larsen & Kristensen 1997 Brain Res. Mol. Brain Res 48: 1-6). It should be noted that PP from the blood has access to this area of the brain since the blood brain barrier is “leaky” in this area where various hormones from the periphery are sensed. Recently it has been argued that part of the effect of PP on food intake is mediated through an action on neurones—especially the POMC/CART neurones in the arcuate nucleus (Batterham et al. 2004 Abstract 3.3 International NPY Symposium in Coimbra, Portugal). PP acts through Y4 receptors for which it has a subnanomolar affinity as opposed to PYY and NPY which have nanomolar affinity for this receptor (Michel et al. 1998 Pharmacol. Rev. 50: 143-150). PP also has an appreciable affinity for the Y5 receptor, but it is not likely of physiological importance in relation to circulating PP due to both lack of access to the cells in the CNS where this receptor especially is expressed and due to the relatively low affinity for PP.

### PP-Fold Peptide Receptors

There are four well established types of PP-fold peptide receptors in man: Y1, Y2, Y4, and Y5 which all recognize NPY1-36 and PYY1-36 with similar affinity. At one time a Y3 receptor type, which might prefer NPY over PYY, was suggested, but today this is not accepted as a real receptor subtype (Michel et al. 1998 Pharmacol. Rev. 50: 143-150). A Y6 receptor subtype has been cloned, which in man is expressed in a truncated form lacking TM-VII as well as the receptor tail and consequently at least on its own does not appear to form a functional receptor molecule.

Y1 receptors—affinity studies suggest Y1 binds NPY and PYY equally well and basically not PP. Affinity for Y1 is dependent on the identities of both end sequences of the PP-fold molecule (NPY/PYY)— for example residues Tyr1 and Pro2 are essential—and it is dependent on the peptide ends being presented in just the right way. In the C-terminal end, where the side-chains of several of the residues are essential, the Y1 receptor—like the Y5 and Y4 receptor but not the Y2 receptor—tolerates certain substitutions in position 34 (normally a Gln)—such as Pro (Fuhlendorff et al. 1990 J. Biol. Chem. 265: 11706-12, Schwartz et al. 1990 Annals NY Acad. Sci. 61: 35-47). Some structure-function studies concerning the requirements of the Y1 and Y2 receptors have been reported (Beck-Sickinger et al. 1994 Eur. J. Biochem. 225: 947-58; Beck-Sickinger and Jung 1995 Biopolymers 37: 123-42; Söll et al. 2001 Eur. J. Biochem. 268: 2828-37).

Y2 receptors—affinity studies suggest Y2 binds NPY and PYY equally well and basically not PP. The receptor requires especially the C-terminal end of the PP-fold peptide (NPY/PYY). Thus, long C-terminal fragments—down to for example NPY13-36 (the whole alpha helix plus the C-terminal hexapeptide)—are recognized with relatively high affinity, i.e. to within ten-fold of the affinity of the full-length peptide (Sheikh et al. 1989 FEBS Lett. 245: 209-14, Sheikh et al. 1989 J. Biol. Chem. 264: 6648-54). Therefore various N-terminal deletions, which eliminate the binding to the Y1 receptor, still preserve some degree of binding to the Y2 receptor. However, the affinity of the C-terminal fragments is reduced—approximately 10 fold as compared to NPY/PYY for even relatively long fragments. The Gln residue in position 34 of NPY and PYY is highly important for the ligand recognition of the Y2 receptor (Schwartz et al. 1990 Annals NY Acad. Sci. 611: 35-47).

Y4 receptors—affinity studies suggest that Y4 binds PP with subnanomolar affinity corresponding to the concentrations found in plasma whereas NPY and PYY are recognized with much lower affinity. Such studies suggest the Y4 receptor is highly dependent on the C-terminal end of the PP-fold peptides, and that relatively short N-terminal deletions impairs the affinities of the ligands. Some structure activity studies concerning the Y4 receptor have been reported (Gehleft et al. 1996 Mol. Pharmacol. 50: 112-18; Walker et al. 1997 Peptides 18: 609-12).

Y5 receptors—affinity studies suggest that Y5 binds NPY and PYY equally well, and also binds PP with lower affinity, which however is below the normal circulating levels of this hormone. PYY3-36 is also recognized well by the Y5 receptor, however this receptor is to a large degree expressed in the CNS where such peptide cannot get access to the receptor readily when administered in the periphery.

PP-fold peptides and analogs of these have been suggested for use in the treatment of obesity and associated diseases, including for example Prader Willi's syndrome, based on the demonstrated effects of certain of the these peptides in animal models and in man and on the fact that obese people have low basal levels of PYY and PP as well as lower meal responses of these peptides (Holst J J et al. 1983 Int. J. Obes. 7: 529-38; Batterham et al. 1990 Nature). Infusion of PP in patients with Prader Willi's syndrome was early on shown to decrease food intake (Berntson et al. 1993 Peptides 14: 497-503) and this effect has been confirmed by infusion of PP in normal human subjects (Batterham et al 2003, Clin. Endocrinol. Metab. 88: 3989-92). PP-fold peptides have also been suggested for the use in for example therapeutic angiogenesis (Zukowska et al. 2003 Trends Cardiovasc Med. 13:86-92) and in inflammatory bowl disease (see for example WO 03/105763).

However, the native PP-fold peptides are not optimal for use as biopharmaceuticals. For example, the full length peptides, PYY1-36 and NPY1-36 react too broadly with all Y receptor types and will therefore cause cardiovascular side effects and, for example, emesis. Moreover, the natural peptides are not optimized for protein stability as they are made to normally act for a relatively short time as a neuropeptide or hormone. The naturally occurring, more Y2 selective peptide, PYY3-36 has for example the draw back that its PP-fold structure is impaired due to the elimination of the important Pro2 of the poly-proline helix, which in the full length peptide interacts with Tyr27 in the amphiphilic helical region of the molecule.

For the treatment of conditions responsive to Y receptor modulation, it would therefore be desirable to use Y receptor PP-fold peptides or PP-fold peptide mimics which were specific for the selected Y receptor intended as target, and which stably preserve elements of the PP-fold structure important for receptor binding. In particular, it would be highly desirable to use such agents which are selective for the Y2 and Y4 receptors over the Y1 receptor. In several conditions, such as obesity and secretory diarrhoea, the use of an agonist for both the Y2 and Y4 receptors is beneficial. Thus a single compound having both of these properties—Y2 and Y4 agonism—would be highly beneficial. However, in the clinical setting it is important that such a compound is not also a significant agonist on the Y1 receptor, because stimulation of the Y1 receptor leads to unwanted side effects such as cardiovascular side effects (for example, increase in blood pressure), and renal side effects (for example natriuresis). There are natural compounds which are selective agonists for the Y2 receptor as opposed to the Y1 receptor, such as PYY3-36, and which have been suggested for treatment of for example obesity. There are also compounds which are selective agonists for the Y4 receptor as opposed to the Y1 receptor, such as the natural peptide PP, which also have been suggested for treatment of obesity. There are also compounds which, for example are combined agonists for the Y1 and the Y2 receptors, such as the natural peptides PYY and NPY. However, no known compound has previously been reported to be a high potency agonist on both the Y2 and the Y4 receptor. Moreover, it has not previously been suggested to use a combined Y2 and Y4 agonist with selectivity towards the Y1 receptor for therapeutic invention.

Some Common Terms Used in this Specification

Affinity: The affinity of a peptide to a specific receptor is given for example as an IC₅₀ value or a Kᵢ or K_{d} value, which in a specific, non-limiting example is determined in an assay, such as a competition binding assay. The IC50 value corresponds to the concentration of the peptide which displaces a—for the given receptor relevant—radioactive ligand used in an amount far less than the Kd for that radioactive ligand to 50%.

Appetite: A natural desire, or longing for food. Increased appetite generally leads to increased feeding behavior.

Appetite Suppressants: Compounds that decrease the desire for food

Binding: A specific interaction between two molecules, such that the two molecules interact. Binding to a receptor can be specific and selective, so that one molecule is bound preferentially when compared to another molecule. Specific binding may be identified by a disassociation constant (K_{d}). This value is dependent on the selectivity of the compound tested. For example, a compound with a K_{d} that is less than 10 nM is generally considered an excellent drug candidate. However, a compound that has a lower affinity, but is selective for the particular receptor, can also be a good drug candidate.

Body Mass Index (BMI): A mathematical formula for measuring body mass, a Iso sometimes called Quetelet's Index. BMI is calculated by dividing weight (in kg) by height² (in meters). The current standards for both men and women accepted as “normal” are a BMI of about 20 kg/m². In one embodiment, a BMI of greater than 25 kg/m² can be used to identify an obese subject. Grade I obesity corresponds to a BMI of 25 kg/m². Grade II obesity corresponds to a BMI of 30-40 kg/m²; and Grade III obesity corresponds to a BMI greater than 40 kg/m² (Jequier 1987 Ain. J. Clin. Nutr. 45:1035-47). Ideal body weight will vary among species and individuals based on height, body build, bone structure, and sex.

Caloric intake or calorie intake: The number of calories (energy) consumed by an individual. In the present context this term is identical to the term “energy intake”.

Cosmetic treatment: The term is intended to denote a treatment that is not for medical purposes, but for improving the well-being of a subject e.g. with respect to the appearance of a subject. Included in the term is treatment of a subject who desires to decrease his weight without necessarily being overweight or obese.

Food intake: The amount of food consumed by an individual. Food intake can be measured by volume or by weight. Included within its meaning is i) food intake as being the total amount of food consumed by an individual, and ii) food intake is the amount of proteins, fat, carbohydrates, cholesterol, vitamins, minerals, or any other food component, of the individual. Accordingly, the term food intake as used in the present context is similar to the term “energy intake”.

Normal Daily Diet: The average food intake for an individual of a given species. A normal daily diet can be expressed in terms of caloric intake, protein intake, carbohydrate intake, and/or fat intake. A normal daily diet in humans generally comprises the following: about 2,000, about 2,400, or about 2,800 to significantly more calories. In addition, a normal daily diet in humans generally includes about 12 g to about 45 g of protein, about 120 g to about 610 g of carbohydrate, and about 11 g to about 90 g of fat. A low calorie diet would be no more than about 85%, and preferably no more than about 70%, of the normal caloric intake of a human individual. In animals, the caloric and nutrient requirements vary depending on the species and size of the animal. For example, in cats, the total caloric intake per kg, as well as the percent distribution of protein, carbohydrate and fat varies with the age of the cat and the reproductive state.

Obesity: A condition in which excess body fat may put a person at health risk (see Barlow and Dietz, Pediatrics 102:E29, 1998, National Institutes of Health, National Heart, Lung, and Blood Institute (NHLBI), Obes. Res. 6 (suppl. 2):51 S209S, 1998). Excess body fat is a result of an imbalance of energy intake and energy expenditure. In one embodiment, the Body Mass Index (BMI) is used to assess obesity. In one embodiment, a BMI of from about 22 kg/m² (i.e. about 10% above the normal value) and up to about 30 kg/m² is regarded as overweight, especially from about 25.0 kg/m² and up to 30 kg/m², while a BMI of 30 kg/m² or more is obese.

Overweight: An individual who weighs more than their ideal body weight. An overweight individual can be obese, but is not necessarily obese. In one embodiment, an overweight individual is any individual who desires to decrease their weight. In another embodiment, overweight individual is an individual with a BMI of a BMI of from about 22 kg/m² (i.e. about 10% above the normal value) and up to about 30 kg/m² is regarded as overweight, especially from about 25.0 kg/m² and up to 30 kg/m². It should be noted that subjects having a BMI that is only slightly higher than that of the normal value (e.g. from about 22 to about 25 kg/m²) very often have a desire to loose weight although this may only be for cosmetic reasons.

Potency: In vitro potency of a compound is defined in terms of EC₅₀ values, i.e. the concentration that leads to 50% of the maximally achievable effect as determined in a for the given receptor relevant signalling assay.

Subject: The subject can be any subject, including both human and veterinary mammalian subjects. Thus, the subject can be a human, or can be a non-human primate, a farm animal such as swine, cattle, sheep and poultry, a sport animal or pet such as dogs, cats, horses, hamsters, and rodents.

Therapeutically effective amount: A dose sufficient to prevent, treat or ameliorate a specific condition or disease and/or to alleviate specific signs or symptoms of a specific condition or disease. The term includes a dose that is sufficient or prevent advancement, or to cause regression of a disorder, or which is capable of relieving a sign or symptom of a disorder, or which is capable of achieving a desired result. In embodiments relating to cosmetic treatment or to treatment of overweight or obesity, a therapeutically effect of a receptor agonist is an amount sufficient to inhibit or halt weight gain, or an amount sufficient to decrease appetite, or an amount sufficient to reduce energy or food intake or increase energy expenditure. The term “cosmetically effective amount” is intended to denote a dose that is sufficient for the subject being treated to achieve a desired effect.

### DETAILED DESCRIPTION OF THE INVENTION

In its broadest aspect, the present invention provides the use of a Y receptor agonist which is selective for the Y2 and Y4 receptors over the Y1 receptor, in the preparation of a composition for treatment of conditions responsive to activation of Y2 and/or Y4 receptors

(a) the said agonist being a PP-fold peptide or PP-fold peptide mimic which has
(i) a C-terminal Y receptor-recognition amino acid sequence represented by —X-Thr-Arg-X³-Arg-Tyr-C(═O)NR¹R² wherein R¹ and R¹ are independently hydrogen or C₁-C₆ alkyl X is Val, Ile, Leu or Ala, and X³ is Gln or Asn, or a conservatively substituted variant thereof in which Thr is replaced by His or Asn and/or Tyr is replaced by Trp or Phe; and/or Arg is replaced by Lys, and
(ii) an N-terminal Y receptor-recognition amino acid sequence represented by H₂N—X¹-Pro-X²—(Glu or Asp)—wherein X¹ is not present or is any amino acid residue, and X² is Leu or Ser or conservative substitutions of Leu or Ser, or
   (b) the said agonist comprising
a C-terminal Y receptor-recognition amino acid sequence as defined in (i) above,
said Y receptor-recognition sequence being fused to an amphiphilic amino acid sequence domain comprising at least one alpha helical turn adjacent the N-terminus of the said hexapeptide sequence,
said turn being constrained in a helical configuration by a covalent intramolecular link, and optionally
an N-terminal sequence which commences with a Y receptor-recognition amino acid sequence as defined in (ii) above.

### Specific Invention-Related Terminology

The agonists with which this invention are concerned are selective for the Y2 and Y4 receptors over the Y1 receptor. In the present context, this condition is fulfilled if the agonist has an IC50 value that is at least 10-fold lower for the Y2 and Y4 receptors than for the Y1 receptor when measured in the affinity assay described herein. In general, with respect to potency, the agonists of the invention also have EC50 values at least 10-fold lower for the Y2 and Y4 receptors than for the Y1 receptor when measured in the potency assay described herein. Some of the preferred agonists of the invention have affinities and potencies at least 100-fold higher for the Y2 and Y4 receptors than for the Y1 receptor.

For the purpose of this specification a PP-fold peptide is a molecule having a 3-D structure which, when mapped onto the original 3-D structure of avian PP as determined by X-ray crystallography (Blundell et al. 1981 Proc. Natl. Acad. Sci. USA 78: 4175-79; Glover et al. 1984, Eur. J. Biochem. 142: 379-85), has domains corresponding to, and substantially aligned as in, the N-terminal polyproline-like helix, the type I beta-turn region, the amphiphilic alpha-helix and the C-terminal hexapeptide domains of the said NPY, PYY and/or PP (FIG. 1). The description of the different domains of the PP-fold peptides as used here thus refers to the original X-ray structure of avian PP (Blundell et al. 1981 Proc. Natl. Acad. Sci. USA 78: 4175-79; Glover et al. 1984, Eur. J. Biochem. 142: 379-85; Schwartz et a/1990).

For the purpose of this specification a PP-fold peptide mimic is a molecule having a 3-D structure which, when mapped onto the original 3-D structure of avian PP, at least has domains corresponding to, and substantially aligned as in, the last turn of the amphiphilic alpha-helix, and the C-terminal hexapeptide domains of PP. PP-fold peptide mimics may also, when mapped as aforesaid, have domains corresponding to one or more of the remaining turns of the amphiphilic alpha helix, the N-terminal polyproline-like helix, and the type I beta-turn region, A peptide mimic need not consist entirely of a sequence of alpha amino acids linked by classical peptide bonds. One or more of the bonds in such a sequence may be replaced by peptidomimetic bonds such as reverse amide, and reduced peptide bonds, so that the peptide mimetic may be regarded as a pseudopeptide sequence. Such bond replacements are capable of conferring resistance to endopeptidase degradation and improving pharmacodynamic properties of the molecule.

Such comparisons of 3-D structure as referred to in the above definitions of “PP-fold peptide” and “PP-fold peptide mimic”, may be made by constructing models of the comparison molecules based on their atomic coordinates as determined by for example X-ray diffraction methods, or by use of one or more of the computer programs, which are commercially available for visualization of the predicted 3-D structure of a molecule from its structural formula, for example: “Maestro Modelling Environment” from Schrödinger Inc, 1500 S.W. First Avenue, Suite 1180 Portland, Oreg. 97201; “Insight II Modeling Environment”, Release 4.0, from Accelrys Inc. San Diego; and “SYBYL® 7.0” from Tripos Inc., 1699 South Hanley Rd., St. Louis, Mo., 63144, USA. It should be noted that the 3-D structure of a PP-fold peptide or PP-fold peptide mimic according to the present invention need not, and generally will not, have an exact correspondence with that of a natural NPY, PPY, or PP. The apparent 3-D structure of a PP-fold peptide or PP-fold peptide mimic will vary depending on the experimental conditions used to study this and especially for smaller peptides may appear more or less unfolded under certain conditions. However, it is sufficient that the PP-fold peptide or PP-fold peptide mimic should have domains corresponding to those PP-fold domains specified above, and that it has the structural elements to adopt an overall shape similar to the native peptide in which the C-terminal and if present N-terminal sequences are normally orientated.

The agonists with which the invention is concerned have a C-terminal Y receptor-recognition sequence. This is a sequence, usually about 5-7 residues long and especially a hexapaptide sequence, located at the C-terminus of the agonist, which when present in a PP-fold peptide or PP-fold peptide mimic, binds to the Y receptor and activates the receptor through that binding interaction alone, or as a result of that binding action and the binding to the Y receptor of an N-terminal Y receptor sequence present in the agonist. An N-terminal Y receptor-recognition sequence is a sequence, usually about 3-5 residues long and especially a 4-residue sequence, located at the N-terminus of the agonist, which when present in a PP-fold peptide or PP-fold peptide mimic, binds to the Y receptor and activates the receptor through a combination of that binding action and the binding to the Y receptor of a C-terminal Y receptor sequence present in the agonist. The classic C- and N-terminal Y receptor-recognition sequences are those found in the natural PP, NPY and PPY peptide sequences, but as will become apparent herein, these classic sequences may be modified to recognise Y2 and Y4 but reduce Y1 recognition.

In this specification, terminology such as “residue corresponding to position N of NPY” is a reference to the amino acid residue of the agonist which, on mapping the 3-D structure of the agonist onto that of NPY, most closely maps to residue number N of NPY. The actual numbering of a particular residue in the specific peptide may vary from N due to the occurrence of, for example, deletions in the specific peptide relative to the natural peptides.

In general, PP-fold or PP-fold mimic agonists with which the invention is concerned will usually have a peptidic backbone or a backbone which is partly peptidic, at least in having a C-terminal amino acid sequence and usually an N-terminal amino acid sequence, although the remainder of the backbone may be a non-peptidic linker radical, for example a straight or branched alkylene chain. Amino acids present in the peptidic portion(s) of the agonists will usually be naturally occurring, especially in C- and N-terminal sequences which interact with the Y2 and Y4 receptors, but non-natural alpha amino acids which preserve the PP-fold and do not prevent Y2 and Y4 receptor binding may also be present.

When the agonists with which the invention is concerned have C- and N-terminal amino acid sequences, the C-termini may be amidated and/or the N-termini may be acylated, to confer resistance to carboxy- and/or aminopeptidases. In fact, the C-terminus of the native NPY, PYY and PP peptides are amidated, so the C-terminal amino acid of an agonist of the invention is also amidated.

In this specification, reference is made to amino acids by their common names or abbreviations, such as valine (Val), leucine (Leu), isoleucine (Ile), methionine (Met), phenylalanine (Phe), asparagine (Asn), glutamic acid (Glu), glutamine (Gln), histidine (His), lysine (Lys), arginine (Arg), aspartic acid (Asp), glycine (Gly), alanine (Ala), serine (Ser), threonine (Thr), tyrosine (Tyr), tryptophane (Trp), cysteine (Cys) and proline (Pro). When referred to by its common name or abbreviation, without specifying its steroisomeric form, the amino acid in question is to be understood as the L-form. Where the D-form is intended, the amino acid will be specifically referred to as such. Occasionally, where the context makes it desirable to do so, the L-form will be specified rather than inferred.

The term “conservative substitution” as used herein denotes that one or more amino acids is replaced by another, biologically similar residue. Examples include substitution of amino acid residues with similar characteristics, e.g. small amino acids, acidic amino acids, polar amino acids, basic amino acids, hydrophobic amino acids and aromatic amino acids. Non-limiting examples of conservative amino acid substitutions suitable for use in the present invention include those in the following Table and analogous substitutions of the original residue by non-natural alpha amino acids which have similar characteristics. For example, in a preferred embodiment of the invention Met residues are substituted with norleucine (Nle) which is a bioisostere for Met, but which—as opposed to Met—is not readily oxidised. Another example of a conservative substitution with a residue normally not found in endogenous, mammalian peptides and proteins would be the conservative substitution of Arg or Lys with for example, ornithine, canavanine, aminoethylcysteine or other basic amino acid. For further information concerning phenotypically silent substitutions in peptides and proteins, see, for example, Bowie et. al Science 247, 1306-1310, 1990.

| Original residue | Conservative substitution |
|---|---|
| Ala | Gly |
| Arg | Lys |
| Asn | Gln, His, Thr |
| Asp | Glu |
| Gln | Asn, His |
| Glu | Asp |
| His | Asn, Gln |
| Ile | Leu, Val |
| Leu | Ile, Val |
| Lys | Arg |
| Met | Leu, Ile |
| Phe | Tyr, Trp, His |
| Ser | Thr, Asn |
| Thr | Ser, Asn, Gln |
| Trp | Tyr, Phe, His |
| Tyr | Trp, Phe, His |
| Val | Ile, Leu |

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), trifluoromethyl, trifluoromethoxy, nitro, nitrile (—CN), oxo, phenyl, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B} or —NR^{A}CONR^{A}R^{B} wherein R^{A} and R^{B} are independently a (C₁-C₆)alkyl group. An “optional substituent” may be one of the foregoing substituent groups.

Unless the context dictates otherwise, references herein to NPY, PYY and PP peptides and their sequences relate to the human forms of those peptides and their sequences. However, other mammalian NPY, PYY and PP peptides will often constitute PP-fold peptide mimics of human NPY, PYY and PP, or conservatively substituted human NPY, PYY or PP, as those terms are used herein.

Type (a) Agonists for Use in Accordance with the Invention

In general, type (a) agonists are PP-fold analogues of NPY, PYY or PP, or PP-fold mimics of NPY, PYY or PP, which have modifications to ensure separation of affinity and potency for Y2 and Y4 receptors relative to the Y1 receptor. Such PP-fold analogues and mimics include peptides having the full complement of PP-fold characteristics (N-terminal polyproline-like helix, beta turn, amphiphilic alpha helix and C-terminal hexapeptide), peptide analogues in which part of the backbone (for example the beta turn residues and adjacent residues) is replaced by a non peptidic spacer chain, and truncated peptides possessing the C-terminal hexapeptide and last turn of the amphiphilic alpha helix, but lacking all or part of the polyproline-like helix and/or beta turn.

In the C-terminal Y receptor-recognition amino acid sequence of the agonists with which the invention is concerned, R¹ and R² are preferably each hydrogen. Preferably also, residue X is Val or Ile. In cases where the agonist of the invention is the PP sequence with C-terminal modifications in accordance with the invention, the residue X may be, for example Leu, Val or Ile In cases where the agonist of the invention is the PYY or NPY sequence with C-terminal modifications in accordance with the invention, the residue X may be, for example Val or Ile.

In one embodiment, the agonists with which the invention is concerned comprise a C-terminal Y receptor-recognition sequence represented by —X^{A}—X-Thr-Arg-X³-Arg-Tyr-C(═O)NR¹R² wherein the residue X^{A} is non-basic and non-acidic, and the sequence —X-Thr-Arg-X³-Arg-Tyr-C(═O)NR¹R² is as defined above. In this case the non-basic and non-acidic amino acid residue X^{A} may be, for example, Leu, Met or Nle Ile, Val, or Ala.

In another embodiment, the agonist comprises a C-terminal undecapeptide represented by —X^{C}-Tyr-X^{B}-Asn-X^{A}—X-Thr-Arg-X³-Arg-Tyr-C(═O)NR¹R² wherein the sequence —X^{A}—X-Thr-Arg-X³-Arg-Tyr-C(═O)NR¹R² is as defined above, X^{C} is Arg or Lys and X^{B} is Ile, Leu or Val. In such class of agonist, the C-terminal undecapeptide sequence is -Arg-Tyr-Leu-Asn-(Leu or Met)-Val-Thr-Arg-Gln-Arg-Tyr-C(═O)NH₂

In different embodiment, the agonist comprises a C-terminal undecapeptide sequence represented by —X^{C}-Tyr-X^{B}-Asn-X^{A}—X-Thr-Arg-X³-Arg-Tyr-C(═O)NR¹R² wherein the sequence —X^{A}—X-Thr-Arg-Gln-Arg-Tyr-C(═O)NR¹R² is as defined above, X^{C} is His, Asn, or Gln and X^{B} is Ile, Leu or Val. In such class of agonist, the C-terminal undecapeptide sequence -His-Tyr-(Ile or Leu)-Asn-Met-Leu-Thr-Arg-Gln-Arg-Tyr-C(═O)NH₂.

In the C-terminal Y receptor-recognition amino acid sequence of the agonist, it is currently preferred that X³ is Gln.

In the N-terminal Y receptor-recognition amino acid sequence of the type (a) agonists with which the invention is concerned, the residue X¹ is preferably Ala, or may be absent, and the residue X² is preferably Leu, Ile, or Ser. Hence one preferred N-terminal sequence is H₂N-Ala-Pro-Leu-Glu-. Another preferred N-terminal sequence is H₂N-Pro-Leu-Glu-.

Type (b) Agonists for Use in Accordance with the Invention

In general, type (b) agonists can be regarded as type (a) PP-fold peptide analogues or PP-fold mimics whose minimum PP-fold structural characteristics (the C-terminal Y-receptor recognition sequence and last turn of the alpha helix) are stabilised by a specified intramolecular covalent link.

Type (b) agonists with which the invention is concerned may be PP-fold mimics which have the N-terminal Y receptor recognition sequence discussed above in relation to the type (a) agonists, or they may be N-terminally truncated PP-fold mimics which have no N-terminal Y receptor recognition sequence of the type defined for the type (a) agonists (although they may have other N-terminal amino acid sequences). Therefore the N-terminal receptor recognition sequence defined for the type (a) agonists is optional in this type (b) class of agonist.

Agonists of this type are characterised by an intramolecular link, which either has no equivalent in the native NPY, PYY or PP peptides or which correspond to or substitute a non-covalent interaction with a covalent link, for example the covalent, disulfide bridge between Cys2 and D-Cys27 in [Cys2,D-Cys27,Gln34]PP (SEQ ID No: 22). As stated, such a link can serve to stabilise essential elements of the PP-fold structure, particularly the mobile C-terminal Y2 recognition sequence and last turn of the alpha helix, and/or the presentation of the N-terminus. In the full length or close to full length peptides, such a link can stabilise the native PP-fold structure as such. This is beneficial in two ways, first it stabilizes the C-terminal part of the amphiphilic alpha-helix and thereby the presentation of the C-terminal Y receptor recognition amino acid sequence in an optimal fashion resulting in improved potency towards the Y receptor; secondly, by stabilizing the whole PP-fold as such the peptide becomes less susceptible to proteolytic degradation since such enzymes often require their target sequences to be found in a rather unfolded fashion (Schwartz et al. 1990). The intramolecular link can also enable Y2/Y4-selective agonists which are heavily modified relative to the structure of the native peptides, for example agonists which lack one or more domains, or parts of domains, found in the native peptides.

One set of agonists of this type (b) has a PP-fold structure in which the helical turn-constraining intramolecular link extends from an amino acid residue in the amphiphilic domain to a linkage point in the N-terminal part of the agonist corresponding to the polyproline domain of a PP-fold peptide which extends antiparallel to the amphiphilic domain. The helical turn-constraining intramolecular link, for example a disulfide or lactam link, may in the foregoing cases extend from an amino acid residue in the amphiphilic domain to one in the polyproline-like domain. Where the agonist is a PP-fold mimic whose first four amino acid residues map to the corresponding N-terminal positions of PP, NPY or PYY, the helical turn-constraining intramolecular link, for example a disulfide or lactam link, may extend from an amino acid residue in the amphiphilic domain to one of those four N-terminal residues, as in [Cys2,D-Cys27,Gln34]PP (SEQ ID No: 22), for example.

In another set of agonists of type (b), the helical turn-constraining intramolecular link extends between residues in the last helical turn of the alpha helix domain, for example a lactam link formed between Lys and Glu residues in the helical turn, or between a residue in the C-terminal Y2 recognition amino acid sequence and a residue in the last helical turn of the alpha helix domain, for example for example a lactam bridge between Lys28 and Glu32 in [Lys28,Glu32]PP25-36 (SEQ ID No: 28).

### Type (a) and (b) Agonists

In one set of PP-fold mimic agonists with which the invention is concerned, of type (a) or (b) wherein the agonist has both a C-terminal and N-terminal Y receptor recognition sequence, the C-terminal sequence may be fused at its N-terminus to an amphiphilic amino acid sequence domain comprising at least one alpha helical turn adjacent the N-terminus of the C-terminal hexapeptide sequence, the said C- and N-terminal amino acid sequences being joined by a linker radical, which may be a straight or branched chain alkylene radical, optionally containing one or more double or triple bonds. For example the C- and N-terminal amino acid sequences of the agonist may be joined by peptide bonds to the carboxyl and amino groups respectively of an amino acid of formula NH₂(CH₂)ₙCO₂H wherein n is from 2 to 12, especially 6, 7, 8, 9 or 10. Thus the agonist may be PP-fold mimic of NPY, PPY or PP having the above defined C-terminal and N-terminal Y recognition sequences with a Cys-Cys bridge as described, but with amino acid residues corresponding to 5-24 of the native peptide replaced with an aminocarboxylic acid having a carbon chain of from 6 to 10 carbon atoms selected from the group consisting of 6-aminohexanoic acid (epsilon-aminocaproic acid), 7-aminoheptanoic acid, 8-aminooctanoic acid, 9-aminononanic acid, and aminodecanoic acid. In specific embodiments, 8-aminooctanoic acids (herein sometimes abbreviated as “Aoc”) are preferred. An example of such an agonist is [Cys2,Aoc5-24,D-Cys27,Gln34]-PP (SEQ ID No: 23).

Some of the type agonists with which the invention is concerned may be regarded either 1) as analogs of NPY or PYY peptides in which residues found in certain key positions in PP but not in the corresponding positions of NPY or PYY have been substituted with those corresponding PP residues or natural or non-natural residues which are structurally similar (a conservative substitution) to those corresponding PP residues, or 2) as analogs of PP in which residues found in certain key positions in NPY or PYY but not in the corresponding positions of PP have been substituted with those corresponding NPY or PYY residues or natural or non-natural residues which are structurally similar to those corresponding NPY or PYY residues. As described above and shown in FIG. 1, the Y receptors recognize residues located in the combined C- and N-terminal ends of the peptides presented by the PP-fold or the PP-fold mimetic. Normally PP peptides are not recognized by the Y2 receptor due to the presence of residues in especially the C-terminal end of PP, which are not compatible with a high affinity binding and potency of the peptide on the Y2 receptor. However, by substitution of one or more residues in the PP molecule, or a PP-fold mimic of PP, with the corresponding residue from either NPY or PYY it is possible to increase the affinity of the peptide on the Y2 receptor without losing affinity and potency on the Y4 receptor or only decreasing the affinity and potency on the Y4 receptor to a relatively small degree.

In the table below are indicated the residues which differ between PP, NPY and PYY in the N-terminal and C-terminal ends, i.e. positions 1, 3, 4, 26, 28, 30, 31, and 34. The desired combined Y2 and Y4 high affinity can be obtained through the above described homologous substitution at one or more of these positions. However, a preferred substitution in PP to obtain high affinity and potency on the Y2 receptor without losing affinity and potency on the Y4 receptor is substitution of Pro34 with Gln or a non-natural residue having similar physicochemical properties. Thus [Gln34]PP is a highly preferred peptide, as are PP analogs or mimics in which Gln34 is combined with substitution of one or more other non-NPY/non-PYY residue in PP with the corresponding or with a physicochemically similar residue or with a non-natural amino acid having similar properties.

However it should be noted that permitted homologous substitutions in accordance with the present invention are those which result in agonists as defined herein, since not all possible substitutions suggested by the table will provide the desired properties of the peptide. For example introduction of Pro in position 34 in PYY or NPY impairs the affinity of the peptides for the Y2 receptor too much for the analog to be useful in the present invention although it provides a high affinity for the Y4 receptor.

| | Position | | | | | | | |
|---|---|---|---|---|---|---|---|---|
| | 1 | 3 | 4 | 26 | 28 | 30 | 31 | 34 |
| PP | Ala | Leu | Glu | Arg | Ile | Met | Leu | Pro |
| NPY | Tyr | Ser | Lys | His | Ile | Leu | Ile | Gln |
| PYY | Tyr | Ile | Lys | His | Leu | Leu | Val | Gln |

Specific Agonists for Use in Accordance with the Invention

Specific examples of agonists for use in accordance with the invention are the following:

### [Gln34] PP (SEQ ID No: 4)

[N—(N′-hexadecanoyl)-gammagluatamoyl-Lys13,Gln34]PP (SEQ ID No: 34)

### [N-(Ala-Arg-Arg-Arg-Ala-Ala-Arg-Ala)₃-Lys18,Gln34]PP (SEQ ID No: 35)

### [N-PEG5000-Lys13,Gln34]PP (SEQ ID No: 36)

### [Ile31,Gln34]PP (SEQ ID No: 5)

### [Val31,Gln34]PP (SEQ ID No: 6)

### [Leu30,Gln34]PP (SEQ ID No: 7)

### [Nle30,Gln34]PP (SEQ ID No: 8)

### [Leu28,Gln34]PP (SEQ ID No: 9)

### [His26,Gln34]PP (SEQ ID No: 10)

### [Ile3,Gln34]PP (SEQ ID No: 11)

### [Ala1,Glu4,Arg26,(Met30 or Nle30)]PYY (SEQ ID No: 12)

[Ala1,Glu4, N—(N′-hexadecanoyl)-gammagluatamoyl-Lys13,Arg26,Nle30]PYY (SEQ ID No: 37)

### [Ala1,Glu4, N-(Ala-Arg-Arg-Arg-Ala-Ala-Arg-Ala)₃-Lys13,Arg26, Nle30]PYY (SEQ ID No: 38)

### [Ala1,Glu4,N-PEG5000-Lys13,Arg26,Nle30]PYY (SEQ ID No: 39)

### [Ala1,Glu4,Arg26(Met30 or Nle30)]NPY (SEQ ID No: 13)

### [Ala1,Glu4]PYY (SEQ ID No: 14) and [Ala1,Glu4]NPY (SEQ ID No: 15)

### [Arg26, (Met30 or Nle30)]PYY (SEQ ID No: 16) and [Arg26, (Met30 or Nle30)]NPY (SEQ ID No: 17)

### [Glu4, (Met30 or Nle30)]PYY (SEQ ID No: 18) and [Glu4, (Met30 or Nle30)]NPY (SEQ ID No: 19)

### [Glu4,Arg26]PYY (SEQ ID No: 20) and [Glu4,Arg26]NPY (SEQ ID No: 21)

### [Cys2,D-Cys27,Gln34]PP (SEQ ID No: 22)

### [Cys2,Aoc5-24,D-Cys27,Gln34]PP (SEQ ID No: 23)

### [Cys2,Lys13,D-Cys27,Gln34]PP (SEQ ID No: 30).

[Cys2, N-(8-(8-gammaglutamoylamino-octanoylamino)-octanoyl)-Lys13,D-Cys27,Gln34]PP (SEQ ID No: 31).

### [Cys2, N-{(Ala-Arg-Arg-Arg-Ala-Ala-Arg-Ala)₃}-Lys13, D-Cys27,Gln34]PP (SEQ ID No: 32).

[Cys2, N-{N′-(21-amino-4,7,10,13,16,19-hexaoxaheneicosanoyl)}-gammaglutamoyl-Lys13,D-Cys27,Gln34]PP (SEQ ID No: 33).
and conservatively substituted analogues thereof.

Particularly preferred Y2/Y4-selective agonists for use in accordance with the invention are [Gln34]-PP (SEQ ID No: 4) and [Ala1,Glu4,Arg26, (Met30 or Nle30)]PYY (SEQ ID No: 12) or conservatively substituted analogues thereof.

Adapted Agonists for Use in Accordance with the Invention

Up to this point, basic Y2/Y4-selective agonists for use in accordance with the invention have been described generally, and specific examples of such agonists have been provided. However, various modifications may be made to such agonists, including the specifically identified agonists, for the purpose of improving their pharmacokinetics, pharmacodynamics and metabolic properties. Such modifications may involve linking the agonist to functional groupings (also known as motifs) known per se in the art of peptidic or proteinaceous pharmaceuticals. Three particular modifications of particular benefit in the case of the agonists with which the invention is concerned, whether of type (a) or (b) are linkage with serum albumin binding motifs, or a glycosaminoglycan (GAG) binding motif, or PEGylation.

Serum-Albumin Binding Motifs

Serum albumin binding motifs are typically lipophilic groups, incorporated to enable a prolonged residence in the body upon administration or for other reasons, which may be coupled in various known ways to peptidic or proteinaceous molecules, for example i) via a covalent linkage to e.g. a functional group present on a side-chain amino acid residue, ii) via a functional group inserted in the peptide or in a suitable derivatized peptide, iii) as an integrated part of the peptide. For example, WO 96/29344 (Novo Nordisk A/S) and P. Kurtzhals et al. 1995 Biochemical J. 312: 725-31 and L. B. Knudsen et al. 2000 J. Med. Chem. 43: 1664-69, describe a number of suitable lipophilic modifications which can be employed in the case of the agonists with which this invention is concerned.

Suitable lipophilic groups include optionally substituted, saturated or unsaturated, straight or branched hydrocarbon groups of from 10 to 24 carbon atoms. Such groups may form, or may form part of, a side chain to the backbone of the agonist, for example by ether, thioether, amino, ester or amide linkage to a side chain of an amino acid residue in the backbone, or to a backbone carbon or a branch from a backbone carbon of a non-peptidic linker radical in the backbone of a PP-fold mimic agonist. The chemistry strategy for attachment of the lipophilic group is not critical, but the following side chains including lipophilic groups are examples which can be linked to a backbone carbon of the agonist, or suitable branch therefrom:
CH₃(CH₂)ₙCH(COOH)NH—CO(CH₂)₂CONH— wherein n is an integer from 9 to 15,
CH₃(CH₂)ᵣCO—NHCH(COOH)(CH₂)₂CONH— wherein r is an integer form 9 to 15,
CH₃(CH₂)_{S}CO—NHCH((CH₂)₂COOH)CONH— wherein s is an integer from 9 to 15,
CH₃(CH₂)ₘCONH—, wherein m is an integer from 8 to 18,
—NHCOCH((CH₂)₂COOH)NH—CO(CH₂)ₚCH₃, wherein p is an integer from 10 to 16,
—NHCO(CH₂)₂CH(COOH)NH—CO(CH₂)_{q}CH₃, wherein q is an integer from 10 to 16,
CH₃(CH₂)ₙCH(COOH)NHCO—, wherein n is an integer from 9 to 15,
CH₃(CH₂)ₚNHCO—, wherein p is an integer from 10 to 18,
—CONHCH(COOH)(CH₂)₄NH—CO(CH₂)ₘCH₃, wherein m is an integer from 8 to 18,
—CONHCH(COOH)(CH₂)₄NH—COCH((CH₂)₂COOH)NH—CO(CH₂)ₚCH₃, wherein p is an integer from 10 to 16,
—CONHCH(COOH)(CH₂)₄NH—CO(CH₂)₂CH(COOH)NH—CO(CH₂)_{q}CH₃, wherein q is an integer from 10 to 16, and
a partly or completely hydrogenated cyclopentanophenanthrene skeleton.

In one chemical synthetic strategy the lipophilic group-containing side chain is a C₁₂, C₁₄, C₁₆ or C₁₈ acyl group, for example a tetradecanoyl group, acylating an amino group present in the side chain of a residue of the backbone of the agonist.

As stated, the modification of agonists for use to provide improved serum protein binding characteristics is a strategy which may be applied in general, and particularly in the case of the specific agonists listed above. Thus for example [Lys11,Gln34]-PP and conservatively substituted analogues thereof may be modified in the above manner for example by tetradecanoyl acylation of the Lys11 or [Lys13,Gln34]PP may be modified to form: [N—(N′-hexadecanoyl)-gammagluatamoyl-Lys13,Gln34]PP and [Ala1,Glu4,Lys13,Arg26,Nle30]PYY may be modified to form: [[Ala1,Glu4,N—(N′-hexadecanoyl)-gammagluatamoyl-Lys13,Arg26,Nle30]PYY.

GAG Binding

As in the case of lipophilic serum binding motifs discussed above, the agonists with which this invention are concerned may be modified by incorporation of the GAG binding motif as, or as part of, a side chain to the backbone of the agonist. Known GAG-binding motifs for incorporation in this way include the amino acid sequences XBBXBX and/or XBBBXXBX, wherein B is a basic amino acid residue and X is any amino acid residue. A plurality, for example three, of such sequences may be incorporated in a concatameric (straight chain) or dendrimeric (branched chain) fashion. Specific concatameric GAG motifs include Ala-Arg-Arg-Arg-Ala-Ala-Arg-Ala-Ala-Arg-Arg-Arg-Ala-Ala-Arg-Ala, and Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala (both of which may, for example be coupled through an amide bond formed between the C-terminus of the concatameric GAG-binding motif and an amino group in the side chain of a backbone amino acid of the agonist, such as the epsilon amino group of Lys18 in the agonist [Lys18,Gln34]PP (SEQ ID No: 24) to form: [N-(Ala-Arg-Arg-Arg-Ala-Ala-Arg-Ala)₃-Lys18,Gln34]PP or Lys13 in the agonist [Ala1,Glu4,Lys13,Arg26,Nle30]PYY to form: [Ala1, Glu4, N-(Ala-Arg-Arg-Arg-Ala-Ala-Arg-Ala)₃-Lys13,Arg26, Nle30]PYY.

Instead of being attached to the agonist as, or as part of a side chain to a backbone residue, the GAG motif may be covalently linked to the C— or (preferably) N-terminus of the agonist, either directly or via a linker radical. Here also the GAG-binding motif may comprise the amino acid sequence XBBXBX and/or XBBBXXBX, wherein B is a basic amino acid residue and X is any amino acid residue, for example the sequence [XBBBXXBX]ₙ where n is 1 to 5, B is a basic amino acid residue and X is any amino acid residue, particularly Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala-[Gln34]PP (SEQ ID No: 26).

The Y2/Y4 selective agonists with which the present invention is concerned are useful, inter alia, for therapeutic angiogenesis. For this use in particular, the agonists preferably comprise a glycosamino glycan (GAG) binding motif as discussed above. Such motifs ensure that the agonists bind to GAGs in the extracellular matrix, and thereby ensures prolonged local exposure of the Y receptors in that tissue. Growth factors, chemokines etc bind to GAGs through patches of basic amino acids, which interact with the acidic sugars of the GAGs. These positively charged epitopes on the growth factors are usually composed of side chains from basic residues, which are not necessarily located consecutively in sequence but are often presented in close proximity by a secondary structural element such as an a-helix or a turn or by the overall three dimensional structure of the protein. Certain GAG-binding, linear sequences, discussed above, have been described, for example XBBXBX and XBBBXXBX where B represents a basic residue (Hileman et al. Bioassays 1998, 20: 156-67). These segments have been shown by circular dichroism to form α-helices upon binding to GAGs. If such sequences are placed for example in a concatameric or dendrimeric construct where for example three such sequences are presented—for example each as a ARRRAARA sequence—the resulting 24-mer peptide—for example ARRRAARA-ARRRAARA-ARRRAARA—ensures a retention in the extracellular matrix similar to high molecular weight polylysine, i.e. it is not washed out during a 4 hour perfusion period (Sakharov et al. FEBS Lett 2003, 27: 6-10).

Thus Growth factors and chemokines are naturally constructed with two types of binding motifs: one binding motif for the receptor through which signal transduction is achieved and one binding motif for GAG's through which attachment and long-lasting local activity is achieved. Peptides such as NPY and PP are neuropeptides and hormones, which are rather rapidly washed out of the tissue and are not optimized for long-lasting local activity. By attaching a GAG-binding motif to a Y2/Y4 selective agonist according to the present invention a bi-functional molecule similar to the growth factors and chemokines is constructed having both a receptor binding epitope in the PP-fold peptide part and a GAG-binding motif. Moreover, by including a GAG-binding motif, the modified peptide also has the property of staying longer in the tissue when injected, for example subcutaneously or intramuscularly or trans-dermally, or in a similar manner delivered into the tissue as such. Thus a GAG modified peptide also constitutes a slow release formulation, which can be highly beneficial to obtain slow release to the circulation and prolonged mode of action.

Another suitable position for introduction of a GAG binding motif—such as a basic concatameric or dendrimeric construct—of the PP-fold peptides is for NPY peptides and analogs thereof, position 14, and for PYY and PP peptides and analogs thereof position 13. For both NPY, PYY and PP type of peptides other preferred postions are position 11 and 18. However, as discussed above, a residue comprising the GAG-binding motif can be introduced at any position in the agonists provided that this introduction is consistent with retention of the PP-fold structure required in the type (a), (b) and (c) agonists with which the invention is concerned, and the required C-terminal Y2-recognition sequence. Thus, a GAG-binding motif could be placed as part of the spacer construct in the analogs in which part of the PP-fold is replaced by a non-peptide spacer.

### PEGylation

In PEGylation, a polyalkyleneoxide radical or radicals, is/are covalently coupled to peptidic or proteinaceous drugs to improve effective half life in the body following administration. The term derives from the preferred polyalkyleneoxide used in such processes, namely that derived from ethylene glycol—polyethyleneglycol, or “PEG”.

A suitable PEG radical may be attached to the agonist by any convenient chemistry, for example via a backbone amino acid residue of the agonist. For instance, for a molecule like e.g. PEG, a frequently used attachment group is the epsilon-amino group of lysine or the N-terminal amino group. Other attachment groups include a free carboxylic acid group (e.g. that of the C-terminal amino acid residue or of an aspartic acid or glutamic acid residue), suitably activated carbonyl groups, mercapto groups (e.g. that of a cysteine residue), aromatic acid residues (e.g. Phe, Tyr, Trp), hydroxy groups (e.g. that of Ser, Thr or OH-Lys), guanidine (e.g. Arg), imidazole (e.g. His), and oxidized carbohydrate moieties.

When the agonist is PEGylated it usually comprises from 1 to 5 polyethylene glycol (PEG) molecules such as, e.g. 1, 2 or 3 PEG molecules. Each PEG molecule may have a molecular weight of from about 5 kDa (kiloDalton) to about 100 kDa, such as a molecular weight of from about 10 kDa to about 40 kDa, e.g., about 12 kDa or preferably no more than about 20 kDa.

Suitable PEG molecules are available from Shearwater Polymers, Inc. and Enzon, Inc. and may be selected from SS-PEG, NPC-PEG, aldehyde-PEG, mPEG-SPA, mPEG-SCM, mPEG-BTC, SC-PEG, tresylated mPEG (U.S. Pat. No. 5,880,255), or oxycarbonyl-oxy-N-dicarboxylmide-PEG (U.S. Pat. No. 5,122,614).

In a particular embodiment the agonist [Lys13, Gln34]PP referred to above may be PEGylated at Lys13 to form: [N-PEG5000-Lys13,Gln34]PP (SEQ ID No: 40) or [Ala1,Glu4,Lys18, Arg26, (Met30 or Nle30)]PYY (SEQ ID No: 25) may be PEGylated at Lys18.

### Serum Albumin, GAG and PEG

Whether the modification to the agonist is attachment of a group to facilitate serum binding, GAG binding or improved stability via PEGylation, the serum albumin binding motif or GAG binding motif, or PEG radical may be, or may form part of, a side chain of a backbone carbon of the agonist corresponding to any of the following positions of PYY or PP: 1, 3, 6, 7, 10, 11, 12, 13, 15, 16, 17, 18, 19, 21, 22, 23, 25, 26, 28, 29, 30 and 32, or corresponding to any of the following positions of NPY: 1, 3, 6, 7, 10, 11, 12, 14, 15, 16, 17, 18, 19, 21, 22, 23, 25, 26, 28, 29, 30 and 32.

### Conjugation to Larger Biomolecules

As described above, It is possible to link certain motifs at various positions in the PP-fold peptide or PP-fold peptide mimic without impairing their high Y receptor affinity (see examples). In a similar way the combined selective Y2-Y4 receptor agonists may be used as fusion proteins where they are linked for example to album in or another protein or carrier molecule which provides beneficial pharmacokinetic or other types of properties such as for example decreased renal elimination. There are multiple chemical modifications and linkers which can be used for such a covalent attachment as known in the art, just as there are multiple proteins or carriers which can be used. Especially covalent attachment of the selective Y2-Y4 peptide agonist to albumin is preferred and at one of the positions in the PP-fold structure, which have been pointed out elsewhere herein in relation to modifications with the various motifs. Such fusion proteins can be produced through various semi-synthetic techniques where the peptide may be made through peptide synthesis as described herein and the biomolecule through recombinant technology. The fusion protein may also be made entirely as a recombinant molecule expressed for example as a precursor molecule extended by a Gly-Lys-Arg sequence, which when expressed as a secretory protein in eukaryotic cells will be cleaved by biosynthetic enzymes and the Gly turned into the carboxyamide on the C-terminal Tyr residue of the C-terminal Y2 receptor recognition sequence.

### Stabilization

As mentioned at various points herein, many of the of the selective Y2-Y4 agonists of the present invention are stabilized in various ways, for example by N-terminal acylation, or by replacement of parts of the agonist backbone with a non-peptidic linker, or by internal cyclizing cross links to stabilize the PP-fold structure. This stabilization in most cases serves two purposes, one being to present the receptor recognition epitopes in an optimal way for the Y2 and Y4 receptors by preserving the PP-fold; is the other being to stabilize the peptide against degradation, i.e. especially proteolytic degradation. This is particularly important when the selective Y2-Y4 agonists are also modified to obtain prolonged half-life, sustained release, and/or long-lasting tissue exposure, by the attachment of the various motifs discussed above. Normally, the peptide agonists will be relatively rapidly eliminated mainly through the kidneys and the protein stability as such may not be a critical issue; however, when the presence of the peptide in various body fluids in one or more ways is prolonged for many hours it is particularly important that the biological activity of the peptide is also kept intact, i.e. that it is stabilized in a form resistant to proteolytic attacks, as described both in general and in relation to the specific examples of selective Y2-Y4 agonist peptides, for example the PP-fold stabilized, cyclic [Cys2,D-Cys27]PP and the various analogs of this. Thus in preferred embodiments of the invention the combined Y2-Y4 selective agonists are both structurally stabilized—through structural alterations some of which are specifically indicated in the examples presented below—and they are decorated with various motifs and/or fused to biomolecules and/or prepared in pharmaceutical manners to obtain sustained release etc. to obtain prolonged receptor exposure.

### Helix Inducing Peptides

Acylation of the N-terminus of the agonists with which the invention is concerned has been mentioned as a means of stabilising the agonist against the action of aminopeptidases. Another stabilising modification involves the covalent attachment of a stabilizing peptide sequence of 4-20 amino acid residues covalently at the N- and/or the C-terminus, preferably the N-terminus. The amino acid residues in such a peptide are selected from the group consisting of Ala, Leu, Ser, Thr, Tyr, Asn, Gln, Asp, Glu, Lys, Arg, His, Met and the like. In an interesting embodiment the N-terminal peptide attachment comprises 4, 5 or 6 Lys residues, for example Lys-Lys-Lys-Lys-Lys-Lys-[Gln34PP} (SEQ ID No: 29). These can be linked at the N-terminus of the PP-fold peptide agonist or they may be placed at the N-terminus of an N-terminally truncated PP-fold mimic agonist or an N-terminally shortened PP-fold mimic agonist, where a spacer peptide has been introduced between the new N-terminus an the stabilizing peptide. A general description of such stabilizing peptide extensions is given in WO 99/46283 (Zealand Pharmaceuticals), which is hereby incorporated by reference.

The receptor agonists with which the invention is concerned may be prepared by well-known methods such as, e.g., a synthetic, semisynthetic and/or recombinant method. The methods include standard peptide preparation techniques such as, e.g., solution synthesis, and solid-phase synthesis. Based on textbook and general knowledge within the field, a person skilled in the art knows how to proceed in order to obtain the agonists and derivatives or modifications thereof.

In the following, the specific agonists according to the invention are described in relation to their molecular pharmacological properties as well as peptide chemical and stability properties.

### [Ile31,Gln34]PP (SEQ ID No: 5)

This peptide is a PP analog designed for high affinity on the Y2 receptor through introduction of Ile in position 31 and Gln in position 34 corresponding to the residue found at these positions in NPY (Fuhlendorff et al. JBC 1990, 265: 11706-12). Receptor recognition profile-[Ile31,Gln34]PP has a single digit nanomolar affinity on the human Y4 receptor (IC50=1.2 nM) very similar to that of PP (IC50=0.49 nM) and [Ile31,Gln34]PP has an affinity for the Y2 receptor of 54 nM but an IC50 of more than 1000 nM on the Y1 receptor—and, accordingly [Ile31,Gln34]PP is a selective Y2-Y4 agonist as compared to the Y1 receptor (Table 1). Affinities for Y receptors were previously reported at a time where only the rat Y4 receptor was available, where [Ile31,Gln34]PP was shown to have an affinity of around 50 nM on the rat Y4 receptor, which is around 100 fold lower than the affinity of PP (Fuhlendorff et al. JBC 1990, 265: 11706-12, Schwartz et al. NYAC, 1990, 611: 35-47). In respect of potency, [Ile31,Gln34]PP displayed an in vitro potency on the Y4 receptor of 0.91 nM (EC50 value) on the Y2 receptor an EC50 of 6.5 nM and on the Y1 receptor an EC50 value of 48 nM (Table 2). Thus, as compared to the previously published affinity data (Fuhlendorff et al. JBC 1990, 265: 11706-12, Schwartz et al. NYAC, 1990, 611: 35-47) it is surprising that [Ile31,Gln34]PP in fact is a single digit potency agonist both on the Y2 and the Y4 receptor as opposed to its double digit potency on the Y1 receptor.

Protein stability—similar to PP1-36.

### [Val31,Gln34]PP (SEQ ID No: 6)

Like [Ile31,Gln34]PP (SEQ ID No: 5) this peptide is a PP analog designed for high affinity on the Y2 receptor but with retained high affinity for the Y4 receptor, however in this case through introduction of Val in position 31 corresponding to the residue found in PYY at this position and again Gln in position 34 corresponding to the residue found at this position in both NPY and PYY. This is a novel compound. Receptor recognition profile-[Val31,Gln34]PP has an affinity of 0.93 nM on the Y4 receptor and 69 nM on the Y2 receptor whereas its affinity cannot be measured on the Y1 receptor even using >1000 nM (Table 1)—thus [Val31,Gln34]PP is a Y2-Y4 selective ligand. In respect of in vitro potency, [Val31,Gln34]PP has an EC50 value of 1.0 nM on the Y4 receptor and 9.9 nM on the Y2 receptor, whereas its potency on the Y1 receptor is 99 nM (Table 2).

Protein stability—similar to PP1-36.

### [Gln34]PP (SEQ ID No: 4)

Like [Ile31,Gln34]PP (SEQ ID No: 5) and [Val31,Gln34]PP (SEQ ID No: 6) this peptide is a PP analog designed for high affinity on the Y2 receptor, however in this case only through introduction of Gln in position 34 corresponding to the residue found at this position in both NPY and PYY. Due to the fact that position 34 is not crucial for the recognition of PP at the Y4 receptor the peptide has dual high affinity on the Y2 and the Y4 receptors. In bovine PP Gln has been introduced in position 34 and it was found to have an affinity on the Y4 receptor very similar to that of bovine PP itself, but this peptide was not tested on the Y2 receptor (Gehlert et al 1996 Mol Pharmacol. 1996 50:112-8). In the present invention the Gln for Pro substitution is made in the human PP1-36 and it is demonstrated that [Gln34]PP surprisingly has a high affinity on the Y2 receptor similar to that of NPY and PYY and that it does not bind to the Y1 receptor. Thus [Gln34]PP (i.e. [Gln34]humanPP1-36) is a novel compound being selective for the Y2 and Y4 receptor as opposed to the Y1 receptor. Receptor recognition profile-[Gln34]PP binds with an affinity of 1.2 nM to the Y4 receptor and with an affinity of 9.0 nM to the Y2 receptor whereas its affinity could not be measured on the Y1 receptor even using >1000 nM of the peptide (Table 1). In respect of in vitro potency, [Gln34]PP shows an EC50 value of 1.1 nM on the Y4 receptor and 3.0 nM on the Y2 receptor, whereas its potency on the Y1 receptor is 388 nM (Table 2).

Protein stability—similar to PP1-36.

In vivo receptor selectivity—In a dose-escalation study [Gln34]PP was administered by intravenous infusion to anesthetized dogs in consecutive 30 minute infusion periods in doses of 0.3, 1, 3, 10, 30 and 100 μg/kg. Plasma levels of [Gln34]PP was measured by radioimmunoassay, which demonstrated a linear dose relationship. At the highest dose a plasma levels of 100 nM was obtained in the dogs. Nevertheless no dose-dependent effect on neither blood pressure nor on heart rate was observed. It is well known that PYY and NPY, which are agonists both on the Y1 and the Y2 receptors, at much lower plasma levels will increase blood pressure. Thus these experiments indicate that [Gln34]PP also in vivo is a highly selective peptide devoid of measurable Y1 receptor activity.

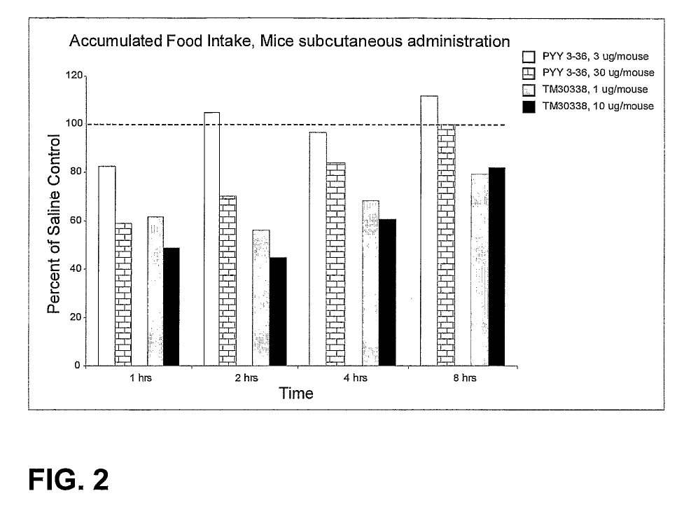

In vivo effect on acute food intake in mice —Either PYY3-36, [Gln34]PP or saline was administered by subcutaneous injections to groups of 8 mice in doses of 3 or 30 μg (PYY3-36) or 1 and 10 μg ([Gln34]PP) per animal after 16 hours of fasting. In FIG. 2 is shown the accumulated food intake of the mice. PYY3-36 at a dose of 30 μg inhibited food intake during the first two hours, but the effect gradually disappeared over the following 2 to 6 hours. [Gln34]PP had a more pronounced and long-lasting inhibitory effect on food intake in mice than PYY3-36. Thus even at a dose of 1 μg a more efficacious inhibition of food intake was observed and the effect lasted for more than 8 hours FIG. 2. Thus [Gln34]PP, which is 10-fold less potent on the Y2 receptor as measured in in vitro assays (EC50 values of 3.0 versus 0.24 for PYY3-36, Table 2) nevertheless appeared to be more potent and to have a more prolonged effect on acute food intake in mice in vivo conceivably due to its effect also as a Y4 agonist.

[Gln34]PP can—in analogy with other peptide examples presented here—be further modified to obtain various for example pharmacokinetic or other purposes while keeping the combined Y2-Y4 over Y1 selectivity. For example Met residues at position 17 and/or 30 can be substituted with for example Leu or Nle, one or more Lys or other attachment prone residues can be introduced at various position for the attachment of various motifs etc. Hereby a group of selective Y2-Y4 agonist peptides can be generated from which compounds can be selected as drugs for treatment of obesity etc. as described elsewhere herein.

### [Leu30,Gln34]PP (SEQ ID No: 7), [Nle30,Gln34]PP (SEQ ID No: 8), [Leu28,Gln34]PP (SEQ ID No: 9), [His26,Gln34]PP (SEQ ID No: 10)

Like [Ile31,Gln34]PP (SEQ ID No: 5) and [Val31,Gln34]PP (SEQ ID No: 6) these peptides are PP analogs designed for high affinity on the Y2 receptor, however in these cases the Pro34 to Gln substitution is combined with substitution of other non-NPY/non-PYY residues in the C-terminal end of PP: a Met30 to Leu (as in both NPY and PYY) or with Ile28 to Leu (as in PYY, NPY has Ile as PP) or with Arg26 to His (as in both NPY and PYY). For example, substitution of Met30 with Leu or Nle has the advantage that this eliminates the possibility of oxidation of the naturally occurring Met residue. [Leu30,Gln34]PP, [Nle30,Gln34]PP, [Leu28,Gln34]PP, and [His26,Gln34]PP are novel compounds

### [Ile3,Gln34]PP (SEQ ID No: 11)

[Ile3,Gln34]PP represents peptides being modified both in the C and N terminal ends in a PP scaffold background to obtain the desired combined Y2-Y4 selectivity towards Y1. Like, for example [Ile31,Gln34]PP and [Val31,Gln34]PP this peptide is a PP analog designed for high affinity on the Y2 receptor, however in this case the Pro34 to Gln substitution is combined with substitution of another non-NPY/non-PYY residues in this case in the N-terminal end of PP: Leu3 to Ile (as in PYY—it is Ser in NPY). [Ile3,Gln34]PP is a novel compound.

### [Ala1,Glu4,Arg26,Met30]PYY (SEQ ID No: 12).

This peptide here represents a group of PYY and NPY analogs in which one or more non-PP residues in the N— and or C-terminal end(s) have been substituted with residue(s) found in PP to obtain the desired combined Y2-Y4 selectivity towards Y1. [Ala1,Glu4,Arg26,Met30]PYY has been designed to obtain a high affinity on the Y4 receptor through the introduction of a total of four PP-like residues in the C-terminal and N-.terminal ends. Introduction of Pro at position 34 is avoided as this strongly impairs the affinity of the peptides for the Y2 receptor although it increases the affinity of the peptides on the Y4 receptor. [Ala1,Glu4,Arg26,Met30]PYY is a novel compound.

Receptor recognition profile-[Ala1,Glu4,Arg26,Met30]PYY displays a high potency in respect of stimulating the human Y2 receptor (EC50=1.0 nM) and the human Y4 receptor (EC50=1.4 nM) whereas it only has a potency of 87 nM on the human Y1 receptor (Table 2).

Protein stability—similar to PYY1-36

[Ala1,Glu4,Arg26,Met30]PYY can—in analogy with other peptide examples presented here—be further modified to obtain various for example pharmacokinetic or other purposes while keeping the combined Y2-Y4 over Y1 selectivity. For example Met residues at position 17 and/or 30 can be substituted with for example Leu or Nle, one or more Lys or other attachment prone residues can be introduced at various position for the attachment of various motifs etc. Hereby a group of selective Y2-Y4 agonist peptides can be generated from which compounds can be selected as drugs for treatment of obesity etc. as described elsewhere herein.

### [Ala1,Glu4]PYY (SEQ ID No: 14), [Arg26,Met30]PYY (SEQ ID No: 16), [Glu4,Met30]PYY (SEQ ID No: 18), [Glu4,Arg26]PYY (SEQ ID No: 20)

These novel peptides together with [Ala1,Glu4,Arg26,Met30]PYY represent a group of peptide agonists based on PYY or NPY in which residues have been substituted in either the C- and/or the N-terminal ends in order to obtain the Y2 plus Y4 as opposed to Y1 receptor selectivity. Hereby and together with the other PP-based peptides a spectrum of peptides are generated from which can be selected peptides having an optimal balance between high Y2 and Y4 affinities and with variable Y1 potencies to be used as an anti-obesity etc. drug.

Receptor recognition profiles—as examples are in Table 1 shown that the affinity of [Arg26,Met30]PYY on the Y1 and Y4 receptors is 0.13 nM and 2.3 nM respectively as opposed to an affinity of 65 nM on the Y1 receptor. As another example, the affinity of PYY is decreased from 16 nM on the Y1 receptor to 367 nM through the Glu for Lys substitution on position 4 and the Arg for His substitution in position 26 in [Glu4,Arg26]PYY while this peptide through these two substitutions has gained Y4 affinity from 30 nM (for PYY) to 3.3 nM for [Glu4,Arg26]PYY thus creating a selectivity window of more than 100-fold (Table 1).

### [Cys2,D-Cys27,Gln34]PP (SEQ ID No: 22)

Here this PP analog represents a group of Y2-Y4 selective agonists in which the increased protein stability of a cyclic [Cys2,D-Cys27]PP peptide is combined with the dual Y2 and Y4 versus Y1 receptor selectivity of [Gln34]PP. This is a novel compound. From the above presented peptides it will be clear that this peptide can be further modified to obtain various for example pharmacokinetic or other purposes while keeping the combined Y2-Y4 over Y1 selectivity. For example Met residues at position 17 and/or 30 can be substituted with for example Leu or Nle, one or more Lys or other attachment prone residues can be introduced at various position for the attachment of various motifs etc. Hereby a group of selective Y2-Y4 agonist peptides can be generated from which compounds can be selected as drugs for treatment of obesity etc. as described elsewhere herein

### [Cys2,Aoc5-24,D-Cys27,Gln34]PP (SEQ ID No: 23)

In this PP analog the dual receptor specificity of [Gln34]PP is built into the small convenient [Cys2,Aoc5-24,D-Cys27]PP type analog of PP. This is a novel compound.

### Clinical Indications

The Y2/Y4-specific agonists with which the invention is concerned are of value in the treatment of conditions responsive to activation of Y2 and/or Y4 receptors. Such conditions include those for which regulation of energy intake or energy metabolism, control of intestinal secretion or induction of angiogenesis, is indicated. For any such use, the agonist may be one which comprises a modification or motif which confers stability towards peptidases, serum protein binding properties, or PEGylation to prolong serum and/or tissue half-life. Especially for induction of angiogenesis, the agonist may comprise a GAG-binding motif to prolong tissue half-life and Y receptor exposure.

Diseases or conditions in which regulation of energy intake or energy metabolism is indicated include obesity and overweight, and conditions in which obesity and overweight are considered contributory factors, such as bulimia, bulimia nervosa, Syndrome X (metabolic syndrome), diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, coronary artery disease, myocardial infarction, peripheral vascular disease. stroke, thromboembolic diseases, hypercholesterolemia, hyperlipidemia, gallbladder disease, osteoarthritis, sleep apnea, reproductive disorders such as polycystic ovarian syndrome, or cancer of the breast, prostate, or colon.

Diseases or conditions in which regulation of intestinal secretion is indicated include various forms of diarrhoea or patients suffering from hyper-secretion from their intestinal stomia.

Diseases or conditions where induction of angiogenesis is indicated include peripheral vascular disease, coronary vascular disease, myocardial infarction, stroke, conditions in which any of the foregoing is considered a contributory factor, wound healing and tissue repair.

### 1. Obesity and Overweight

PYY3-36 has been shown to decrease appetite, food intake and body weight in various rodents when administered peripherally (Batterham et al. Nature 2002, 418: 595-7; Challis et al. BBRC November 2003, 311: 915-9) as well as to decrease appetite and food intake in man also when administered peripherally (Batterham et al 2002). The animal data including studies in receptor knock out animals strongly indicate that this effect of PYY3-36 is mediated through Y2 receptors and through NPY/AgRP and POMC neurones in the arcuate nucleus. PYY levels and the PYY food responses have often been reported to be lower in obese subjects and correlates inversely with their BMI. Importantly, obese subject are not resistant to the effect of PYY as infusion of PYY3-36 for 90 minutes decreases food intake in obese subjects in a similar long lasting fashion (Batterham et al. 2003, NEJM 349: 941-48).

Much evidence from recent rodent studies has accumulated showing that PP is a powerful and efficient anorexigenic peptide when administered peripherally (Asakawa et al. Peptides 1999, 20; 1445-8; Katsuura et al. Peptides 2002, 23: 323-9; Asakawa et al. Gastroenterology 2003, 124: 1325-36). Since PP has no effect on appetite, food intake etc. in Y4 knock out animals it is very likely that PP acts through the Y4 receptor to reduce appetite and food intake (Batterham et al. 2004 abstract S3.3 from International NPY symposium in Coimbra Portugal). PP also had effect on food intake in diet induced obese animals. PP receptors have been found especially in the brain stem in vagal motor neurones and in the nucleus tractus solitarius (NTS) both of which are areas where the blood brain barrier is not efficient and where circulating hormones such as PP can get access to the neurones. Thus it is very likely that the Y4 receptors in the NTS in the brain stem are a major target through which PP acts to suppress appetite and food intake. However, recent evidence also points to the possibility that PP may also act through Y receptors in the arcuate nucleus conceivably on the POMC and perhaps also the NPY/AgRP neurones (Batterham et al. Coimbra NPY meeting abstract S3.3). Low levels of PP are found in obese subjects especially Prader-Willi syndrome (Zipf et al. J. C. E. M. 1981, 52: 1264-6, Hoist et al 1983, Int. J. Obes. 7: 529-38, Glaser et al Horm. Metab. 1988, 20: 288-92) and high PP levels are found in patients with anorexia nervosa. Importantly, infusion of PP in man decreases appetite and food intake for up to 24 hours (Batterham et al. JCEM 2003, 88: 3989-92). Thus, the effect of PP on food intake was observed after the PP levels in the circulation had returned to normal levels. Such long lasting effects on appetite etc, is well know also from ICV injection of especially AgRP. Importantly infusion of PP has also been shown to decrease food intake in morbidly obese patients with Prader Willi syndrome (Berntson et al 1993 Peptides 14: 497-503).

Due to the fact that PYY acting through the Y2 receptors conceivably mainly in the arcuate nucleus—inhibiting the stimulatory NPY/AgRP neurones—and PP acting through Y4 receptors mainly in the area postreama and NTS in the brain stem but also in the arcuate nucleus—but apparently stimulating the inhibitory POMC neurones (Batterham et al 2004 International NPY symposium, Coimbra abstract S3.3), the combined effect of a Y2 agonist and a Y4 agonist will have an additive or even a synergistic effect, i.e. that a more efficient effect is achieved from a combined treatment than from each of the two treatments by them selves.

PYY itself is know to be extremely emetic when administered peripherally, in fact PYY was discovered—for “the second time”—in 1989 as the biologically active entity in a chromatographic fraction of an intestinal extract causing dogs to vomit (Harding and McDonald 1989 Peptides 10: 21-24). It was concluded that PYY was the most potent, circulating emetic peptide identified and that this effect was mediated through area postreama known to have a leaky blood brain barrier. It has also been reported that PYY3-36 can cause nausea when administered peripherally to human subjects (Nastech press release 29 Jun. 2004). From a physiological point of view it appears logically that PYY—and its biologically active conversion product—which normally first is secreted in large amounts during a very large meal or when food is dumped into the lower intestine due to various surgical procedures, is able to cause emesis and vomiting to relieve the subject from a situation of overt overeating. Interestingly, it was noted in that paper that PP given in similar doses did not cause vomiting in these dogs (Harding and McDonald 1989). PP acts through Y4 receptors also located in the area postreama of the brain stem—but does not cause emesis or vomiting. Large doses of a combined Y2-Y4 agonist peptides such as [Gln34]PP may be administered to animals such as cyno monkeys reaching plasma levels of 12-13.000 nM without observing any vomiting of the animals or evidence of GI-tract side effects. This is surprising since [Gln34]PP has a relatively high potency on the Y2 receptor for which PYY3-36 is totally selective. Thus, surprisingly the combined Y2-Y4 selective agonist does not cause emesis to the same degree as the selective Y2 agonist —PYY3-36 compound—does. Apparently, the Y4 receptor activation—conceivably in the area postreama-prevents the emetic effect of the Y2 activation form the same compound. This property of a combined Y2-Y4 selective agonist is of great benefit for the treatment of obesity and associated conditions.

Hence, the Y2/Y4 selective agonists with which the invention is concerned are suitable for use in a subject, such as a mammal including a human, in order to regulate the energy intake. Accordingly, the invention relates to methods for altering energy intake, food intake, appetite, and energy expenditure. A method is disclosed herein for reducing energy or food intake by administering to a subject a cosmetically or therapeutically effective amount of such an agonist. In one embodiment, administration of the receptor agonist results in a decrease in the amount, either the total weight or the total volume or calorie content of the food. In another embodiment, it may result in a decrease of the intake of a food component, such as a decrease in the ingestion of lipids, carbohydrates, cholesterol, or proteins. In any of the methods disclosed herein, the preferred compounds that have been discussed in details herein could be administered. In an additional embodiment, a method is disclosed herein for reducing appetite by administering a therapeutically effective amount of such an agonist. Appetite can be measured by any means known to one of skill in the art.

For example, decreased appetite can be assessed by a psychological assessment. In such an embodiment, administration of the receptor agonist results in a change in perceived hunger, satiety, and/or fullness. Hunger can be assessed by any means known to one of skill in the art. In one embodiment, hunger is assessed using psychological assays, such as by an assessment of hunger feelings and sensory perception using e.g. a questionnaire.

In a further embodiment, a method is disclosed herein for decreasing the motility of the upper GI tract as for example decreasing gastric emptying. The method includes administering a therapeutically effective amount of such an agonist thereof to the subject, thereby decreasing GI-tract motility. It is well known that compounds which decrease gastric emptying will have a beneficial effect in also decreasing food intake as the subject is feeling more full or satiated. Both PYY3-36, the prototype Y2 agonist, and PP, the prototype Y4 agonists are know to decrease gastric emptying. A combined Y2-Y4 agonist has an additive or even a synergistic effect in inhibiting upper GI-tract motility.

In a further embodiment, a method is disclosed herein for altering energy metabolism in a subject. The method includes administering a therapeutically effective amount of such an agonist thereof to the subject, thereby altering energy expenditure. Energy is burned in all physiological processes. The body can alter the rate of energy expenditure directly, by modulating the efficiency of those processes, or changing the number and nature of processes that are occurring. For example, during digestion the body expends energy moving food through the bowel, and digesting food, and within cells, the efficiency of cellular metabolism can be altered to produce more or less heat. In a further embodiment a method is disclosed herein for any and all manipulations of the arcuate circuitry described in this application, which alter food intake coordinately and reciprocally alter energy expenditure. Energy expenditure is a result of cellular metabolism, protein synthesis, metabolic rate, and calorie utilization. Thus, in this embodiment, peripheral administration results in increased energy expenditure, and decreased efficiency of calorie utilization. In one embodiment, a therapeutically effective amount of a receptor agonist according to the invention is administered to a subject, thereby increasing energy expenditure.

In several embodiments both relating to the therapeutic use and to the cosmetic use, a Y2/Y4 selective agonist can be used for weight control and treatment, reduction or prevention of obesity, in particular any one or more of the following: preventing and reducing weight gain; inducing and promoting weight loss; and reducing obesity as measured by the Body Mass Index. As mentioned above, the invention also relates to the use of a Y2/Y4 selective agonist for controlling any one or more of appetite, satiety and hunger, in particular any one or more of the following: reducing, suppressing and inhibiting appetite; inducing, increasing, enhancing and promoting satiety and sensations of satiety; and reducing, inhibiting and suppressing hunger and sensations of hunger. The disclosure further relates to the use of a Y2/Y4 selective agonist in maintaining any one or more of a desired body weight, a desired Body Mass Index, a desired appearance and good health.

In a further or alternative aspect, the invention relates to a method for the treatment and/or prevention of reduced energy metabolism, feeding disorders, appetite disorders, overweight, obesity, bulimia, bulimia nervosa, Syndrome X (metabolic syndrome), or complications or risks associated thereto including diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, congestive heart failure, stroke, myocardial infarct, thromboembolic diseases, hypercholesterolemia, hyperlipidemia, gallbladder disease, osteoarthritis, sleep apnea, reproductive disorders such as polycystic ovarian syndrome, cancers of the breast, prostate, and colon, the method comprising administering to a subject such as a mammal including a human, an effective dose of one or more of a Y2/Y4 selective agonists as described herein.

### 2. Intestinal Hypersecretion

Both NPY and PYY are known to have anti-secretory effects on both the small and large intestine. Through studies on isolated human colonic tissue it was demonstrated that this effect is mediated through both Y1 and Y2 receptors and by use of TTX it was shown that a major part of the Y2 component was mediated through a neuronal component (Cox & Tough 2001 Br. J. Pharmacol. 135: 1505-12). PP also has a strong anti-secretory effect and this appears to be mediated through the Y4 receptors located on the epithelial cells and not through a neuronal mechanism (Cox & Tough 2001). Thus, due to the similar effect but mediated through different mechanisms a combined Y2-Y4 agonist will have an additive or even a synergistic anti-secretory effect on the GI-tract. It has been shown in vivo that peripheral administration of PYY can cause a long-lasting reduction in intestinal secretion induced by vasoactive intestinal polypeptide in human subjects with ileostomies (Playford et al 1990 Lancet 335: 1555-57). It was concluded that PYY could be a therapeutic agent against diarrhoea, however, for example the natriuretic and hypertensive effects of the combined Y1 and Y2 agonist effects of the peptide has prevented this. The combined selective Y2-Y4 agonists of the present invention are particularly useful for the treatment or protection against hyper secretion of the GI-tract including various forms of diarrhoea whether or not they directly are caused by hyper-secretion. One particularly interesting indication is the hyper-secretion observed in patients with ileostomia, who often are losing large amounts of fluid.

### 3. Therapeutic Angiogenesis

A number of in vitro studies on effects on growth of vascular smooth muscle cells, hyperthrophy of ventricular cardiomyocytes as well as endothelial cell proliferation and migration have suggested that NPY may act as an angiogenic factor (Zukowska-Grojec et al. 1998 Circ. Res. 83: 187-95). Importantly, in vivo studies using both the mouse corneal micropocket model as well as the chick chorioallantoic membrane (CAM) assay has confirmed that NPY is a potent angiogenic factor which gives rise to vascular tree-like structures showing vasodilation as observed otherwise only with fibroblast growth factor-2 (FGF-2) and not for example vascular endothelial growth factor (VEGF) angiogenic structures (Ekstrand et al. 2003 PNAS 100: 6033-38). In the developing chick embryo NPY induced vascular sprouting from preexisting blood vessels. The effect of NPY was not observed in Y2 receptor knock out animals indicating that the Y2 receptor is responsible for the angiogenic effect of NPY (Ekstrand et al 2003). This notion is also supported by observations that the Y2 receptor is highly upregulated in ischemic vessels and the enzyme which generates the endogenous, selective Y2 ligand PYY3-36, dipentidylpeptidase-IV is also highly upregulated. The peptides of the present invention are all Y2 agonist having at least the additional property of also being Y4 agonists and being selective in respect of being poor Y1 agonists. Thus, the Y2 agonist property of these peptides makes them useful as therapeutic angiogenic agents and the Y4 agonism is beneficial in reducing or eliminating the unwanted emetic or nausea promoting effect known to be associated with high plasma levels of Y2 agonists.

In various cardiovascular diseases such as atherosclerosis for example in peripheral vessels as well as in coronary vessels is it contemplated that induction or angiogenesis would be beneficial. Also induction of angiogenesis is believed to be beneficial for securing reperfusion after myocardial infarction. Especially FGF-2 has been proposed to be an efficient agent for induction of angiogenesis in patients with cardiovascular diseases. However, like most other angiogenic factors FGF-2 is a growth factor and has the potential of stimulating tumor growth also by providing angiogenesis. As presented above, NPY acting through Y2 receptors induces neovascularization of a similar type as induced by FGF-2, however NPY is a neuropeptide and not a classical growth factor and has not been implicated in inducing tumor growth. Thus, a Y2 agonist is a useful agent for therapeutic angiogenesis. However, it is particularly important for this use that the agonist does not show Y1 receptor agonism because this will give unwanted cardiovascular effects. This means the peptides with which the invention is concerned, which are e Y2 selective receptor agonists or which are especially useful therapeutic agents also in respect of inducing angiogenesis. They are particularly useful when modified to attach a GAG binding motif, as discussed above. To elaborate further: The action of FGF-2 as that of most other classical growth factors is partly mediated or controlled through their binding to glycosaminoglycans (GAG) in the extracellular matrix. This binding to GAGs secures that the angiogenic factor acts in an appropriate spatial and temporal fashion and that it is not washed out of the tissue rapidly. For the use of small peptides and peptide mimics such as the ones described in the present invention in therapeutic angiogenesis this is particularly important. Thus, in a preferred embodiment of the invention the peptides incorporate one or more GAG binding motif, which secures that they attach to GAGs in the extracellular matrix in order to induce optimal angiogenesis after administration. This can for example be by intravenous or intraarterial administration or for example direct administration into the coronary arteries in order to induce cardiac angiogenesis during coronary artery disease and/or post acute myocardial infarction. Similarly such a compound can be administered through intra arterial injection in the femoral artery for treatment of peripheral vascular disease. It can also be for example topical local administration to skin lesions in order to promote improved wound healing. A prolonged Y receptor exposure efficient in inducing angiogenesis can also be obtained by using a peptide according to the present invention modified with a serum albumin binding motif.

Thus in a preferred embodiment of the invention the Y2/Y4 selective agonists comprise a GAG-binding motif, which is placed in a position where it does not impair the stability of the peptide or impair the potency and selectivity of the peptide. Accordingly, in one embodiment the invention relates to the use a Y2/Y4 selective receptor agonist modifying disturbances in the angiogenesis system, especially for inducing angiogenesis such as angiogenesis associated with diseases or conditions such as e.g., cardiovascular diseases including peripheral vascular disease with symptoms such as cladicatio intermittens, coronary artery disease and myocardial infarction; tissue repair processes including wound healing in the skin, inflammatory conditions including inflammatory conditions in the gastrointestinal tract such as, e.g., ulcers, colitis, inflammatory bowel disease, Crohns disease etc.

A specific embodiment is to use the receptor agonist for inducing angiogenesis in a heart or in a blood vessel, or in a tissue such as a mucosal tissue including the gastro-intestinal mucosa and the skin.

### 3. Wound Healing

In animals where the Y2 receptor has been selectively eliminated through the deletion of its gene it has been reported that wound healing is impaired and that the associated neo-vascularization is impaired (Ekstrand et al. 2003 PNAS 100: 6033-38). Thus the selective Y2-Y4 agonists of the present invention are useful to improve wound healing through their Y2 agonist property. The peptides can for this indication be administered in various ways including parenteral administration. However, a preferred route of administration is topical application e.g. in the form of a solution, dispersion, powders, sticks, creme, ointment, lotion, gel, hydrogel, transdermal delivery system including patches and plasters, etc. For topical administration they can be used as such. However, in a preferred embodiment of the invention, the peptides have been modified with one or more of the GAG-binding motifs described herein to ensure a long lasting, local effect of the peptide through binding to GAGs in the tissue.

### 4. Inflammatory Bowel Disease

PYY has previously been described for the prevention and/or treatment of inflammatory bowel disease; see WO 03/105763 to Amylin Pharmaceuticals, Inc, which is hereby incorporated by reference. Therefore the agonists with which the invention is concerned are effective in the treatment or prevention of inflammatory bowel disease as well. Accordingly, the present invention also relates to the use of the agonists described herein for such medical use. In an interesting embodiment, the peptides comprise one or more GAG-binding motifs, cf. above.

### Additional Comments Concerning Administration of Y2/Y4 Agonists for the Treatment or Prevention of Obesity and Related Diseases

During a meal a large repertoire of gastrointestinal hormones and neurotransmitter systems are activated in a carefully concerted, sequential and overlapping manner. Moreover, food components influence not only the secretion of GI hormones and the activity of various afferent neuronal pathways but these food components after absorption also influence various hormones and centers in the CNS directly. Thus the regulation of food intake and energy expenditure is a highly complex and multifaceted process. In view of this it is surprising that certain hormones such as Y2 agonists can in fact substantially affect the system when administered in a way which results in, for example only 3-4 times the plasma levels which are achieved during a meal.

Part of the problem is that administrations of such compounds—combined Y2-Y4 agonists—will have optimal effect if the compounds are given in the fasting state in an effective dose as described. If the Y2-Y4 agonists are given in a situation where the various hormonal and neuronal systems are active due to the presence of food components in the GI tract or the expectation of a meal, the effect is not seen or a much smaller effect is observed. Thus, in a preferred embodiment of the invention the combined Y2-Y4 agonist is administered in the fasting state in an effective dose either subcutaneously, nasally or through other means as described elsewhere herein. In the present context, the term “fasted state” means that the subject has not eaten any food or drink within at least the last 2 hours before administration of the combined Y2-Y4 receptor agonist such as, e.g., within at least the last 3 hours, within at least the last 4 hours, within at least the last 5 hours, within at least the last 6 hours, within at least the last 7 hours, within at least the last 8 hours, within at least the last 9 hours, within at least the last 10 hours, within at least the last 11 hours or within at least the last 12 hours before dosing.

In a subgroup of the population, the combined Y2-Y4 agonists may not have the intended action due to genetic variations such as polymorphisms in the Y4 receptor gene. Loss of function mutations in these receptors are likely to be associated with obesity. Thus, in a preferred embodiment of the invention an analysis of the Y4 receptor gene of the subject to be treated is performed in order to probe for polymorphisms/mutations in these genes and identification of such polymorphisms. Based on such an analysis an optimal treatment of the subjects can be made. For example, only subjects with normal genotype or with polymorphisms, which do not affect the function of Y4 agonists including Y2-Y4 combined agonists, should be treated with such agonists. Another possibility is to increase the dose of the combined Y2-Y4 agonist in subjects who express an impaired receptor in order to ensure an optimal effect of the drug. In the case where the obesity of a subject is caused by an impairment in the function of the Y4 receptor it could be argued that treatment with a—for example large doses—of a combined Y2-Y4 agonist is a form of replacement therapy—provided that at least some of the relevant receptor function is still left—for example in heterozygote patients. In a situation where it is not possible or not financially suitable to perform such a genetic analysis the use of a selective, combined Y2-Y4 agonist is particularly useful as it—due to its dual effect on two different targets—will be efficacious even in the cases where one of the targets are non-function or have reduced functionality due to genetic polymorphism.

An acute test may be performed to ensure that these compounds have the intended effect in the subject to be treated before a chronic treatment is started, ensuring that only subjects who are susceptible to treatment are treated.

The selective Y2-Y4 agonists of the invention may be combined in the treatment of obesity, diabetes and related diseases with the use of various other drugs targeting appetite and energy expenditure, this includes but is not limited to drugs such as GI-tract lipase inhibitors, neurotransmitter reuptake inhibitors, cannabinoid receptors antagonists and inverse agonists, as well as other types of neurotransmitter—including but not limited to 5HT receptors—and/or hormone—including but not limited to GLP-1, MC4, MC3—receptor agonist or antagonists. Due to the fact that the selective Y2-Y4 agonists are targeting a homeostatic regulatory mechanism in the communication between the GI-tract and the CNS—i.e. the Y2 and the Y4 receptors normally targeted by the satiety mediating hormone PYY from the gut and PP from the pancreas—it is particularly beneficial to combine the treatment with the combined Y2-Y4 selective agonists with the treatment with a drug targeting a central, hedonic mechanism in the regulation of appetite and energy expenditure, such as the CBI receptors, for example being part of the reward system. Thus, the use of selective Y2-Y4 agonists in the treatment of obesity and related diseases in combination with a CB1 antagonist is a preferred embodiment of this invention.

### Dosages

The therapeutically effective amount of a Y2/Y4 receptor agonist according to the invention will be dependent on specific agonist employed, the age, weight and condition of subject being treated, the severity and type of the condition or disease being treated, the manner of administration and the strength of the composition applied. For example, a therapeutically effective amount of a Y2/Y4 receptor agonist thereof can vary from about 0.01 μg per kilogram (kg) body weight to about 1 g per kg body weight, such as about 1 μg to about 5 mg per kg body weight, or about 5 μg to about 1 mg per kg body weight. In another embodiment, the receptor agonist is administered to a subject at 0.5 to 135 picomole (pmol) per kg body weight, or about 72 pmol per kg body weight. In one specific, non-limiting example from about 5 to about 50 nmol is administered as a subcutaneous injection, such as from about 2 to about 20 nmol, or about 1.0 nmol is administered as a subcutaneous injection. The exact dose is readily determined by one skilled in the art based on the potency of the specific compound (such as the receptor agonist) utilized, the age, weight, sex and physiological condition of the subject. The dose of an agonist can be a molar equivalent of the therapeutically effective dose of PYY3-36. The amounts can be divided into one or several doses for administration daily, every second day, weekly, every two weeks, monthly or with any other suitable frequency. Normally, the administration is once or twice daily.

### Methods of Administration

The agonist can be administered by any route, including the enteral (e.g. oral administration) or parenteral route. In a specific embodiment, the parenteral route is preferred and includes intravenous, intraarticular, intraperitoneal, subcutaneous, intramuscular, intrasternal injection and infusion as well as administration by the sublingual, transdermal, topical, transmucosal including nasal route, or by inhalation such as, e.g., pulmonary inhalation. In specific embodiments, the subcutaneous and/or the nasal administration route is preferred.

The receptor agonists can be administered as such dispersed in a suitable vehicle or they can be administered in the form of a suitable pharmaceutical or cosmetic composition. Such compositions are also within the scope of the invention. In the following are described suitable pharmaceutical compositions. A person skilled in the art will know how that such composition may also be suitable for cosmetic use or he will know how to adjust the compositions to cosmetic compositions by use of suitable cosmetically acceptable excipients.

### Pharmaceutical Compositions

The receptor agonists (also denoted “compounds”) according to the invention for use in medicine or cosmetics are normally presented in the form of a pharmaceutical composition comprising the specific compound or a derivative thereof together with one or more physiologically or pharmaceutically acceptable excipients.

The compounds may be administered to an animal including a mammal such as, e.g., a human by any convenient administration route such as, e.g., the oral, buccal, nasal, ocular, pulmonary, topical, transdermal, vaginal, rectal, ocular, parenteral (including inter alia subcutaneous, intramuscular, and intravenous cf. above), route in a dose that is effective for the individual purposes. A person skilled in the art will know how to chose a suitable administration route. As mentioned above, the parenteral administration route is preferred. In a specific embodiment, the receptor agonists are administered subcutaneously and/or nasally. It is well known in the art that subcutaneous injections can be easily self-administered.

A composition suitable for a specific administration route is easily determined by a medical practitioner for each patient individually. Various pharmaceutically acceptable carriers and their formulation are described in standard formulation treatises, e.g., Remington's Pharmaceutical Sciences by E. W. Martin.

The pharmaceutical composition comprising a compound according to the invention may be in the form of a solid, semi-solid or fluid composition. For parenteral use, the composition is normally in the form of a fluid composition or in the form of a semi-solid or solid form for implantation.

Fluid compositions, which are sterile solutions or dispersions can utilized by for example intravenous, intramuscular, intrathecal, epidural, intraperitoneal or subcutaneous injection of infusion. The compounds may also be prepared as a sterile solid composition, which may be dissolved or dispersed before or at the time of administration using e.g. sterile water, saline or other appropriate sterile injectable medium.

The fluid form of the composition may be a solution, an emulsion including nano-emulsions, a suspension, a dispersion, a liposomal composition, a mixture, a spray, or a aerosol (the two latter types are especially relevant for nasal administration).

Suitable mediums for solutions or dispersions are normally based on water or pharmaceutically acceptable solvents e.g. like an oil (e.g. sesame or peanut oil) or an organic solvent like e.g. propanol or isopropanol. A composition according to the invention may comprise further pharmaceutically acceptable excipients such as, e.g., pH adjusting agents, osmotically active agents e.g. in order to adjust the isotonicity of the composition to physiologically acceptable levels, viscosity adjusting agents, suspending agents, emulsifiers, stabilizers, preservatives, antioxidants etc. A preferred medium is water.

Compositions for nasal administration may also contain suitable non-irritating vehicles such as, e.g., polyethylene glycols, glycofurol, etc. as well as absorption enhancers well known by a person skilled in the art (e.g. with reference to Remington's Pharmaceutical Science)

For parenteral administration, in one embodiment the receptor agonists can be formulated generally by mixing it at the desired degree of purity, in a unit dosage injectable form (solution, suspension, or emulsion), with a pharmaceutically acceptable excipient or carrier, i.e., one that is non-toxic to recipients at the dosages and concentrations employed and is compatible with other ingredients of the composition.

Generally, the formulations are prepared by contacting the receptor agonist uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. Due to the amphiphatic nature of the peptides described herein suitable forms also include micellar formulations, liposomes and other types of formulations comprising one or more suitable lipids such as, e.g., phospholipids and the like.

Preferably, they are suspended in an aqueous carrier, for example, in an isotonic buffer solution at a pH of about 3.0 to about 8.0, preferably at a pH of about 3.5 to about 7.4, 3.5 to 6.0, or 3.5 to about 5. Useful buffer substances include acetate, citrate, phosphate, borate, carbonate such as, e.g., sodium citrate-citric acid and sodium phosphate-phosphoric acid, and sodium acetate/acetic acid buffers.

The compositions may also be designed to controlled or prolonged delivery of the receptor agonist after administration in order to obtain a less frequent administration regimen. Normally a dosage regimen including 1-2 daily administrations is considered suitable, but within the scope of the present invention is also included other administration regimens such as, e.g., more frequent and less frequent. In order to achieve a prolonged delivery of the receptor agonist, a suitable vehicle including e.g. lipids or oils may be employed in order to form a depot at the administration site from which the receptor agonist is slowly released into the circulatory system, or an implant may be used. Suitable compositions in this respect include liposomes and biodegradable particles into which the receptor agonist has been incorporated.

In those situations where solid compositions are required, the solid composition may be in the form of tablets such as, e.g. conventional tablets, effervescent tablets, coated tablets, melt tablets or sublingual tablets, pellets, powders, granules, granulates, particulate material, solid dispersions or solid solutions.

A semi-solid form of the composition may be a chewing gum, an ointment, a cream, a liniment, a paste, a gel or a hydrogel.

Other suitable dosages forms of the pharmaceutical compositions according to the invention may be vagitories, suppositories, plasters, patches, tablets, capsules, sachets, troches, devices etc.

The dosage form may be designed to release the compound freely or in a controlled manner e.g. with respect to tablets by suitable coatings.

The pharmaceutical composition may comprise a therapeutically effective amount of a compound according to the invention.

The content of a compound of the invention in a pharmaceutical composition of the invention is e.g. from about 0.1 to about 100% w/w of the pharmaceutical composition.

The pharmaceutical compositions may be prepared by any of the method well known to a person skilled in pharmaceutical formulation.

In pharmaceutical compositions, the compounds are normally combined with a pharmaceutical excipient, i.e. a therapeutically inert substance or carrier.

The carrier may take a wide variety of forms depending on the desired dosage form and administration route.

The pharmaceutically acceptable excipients may be e.g. fillers, binders, disintegrants, diluents, glidants, solvents, emulsifying agents, suspending agents, stabilizers, enhancers, flavours, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

### Synthesis

Peptidic agonists of the invention may be synthesized by solid phase peptide synthesis, using either an automated peptide synthesizer, or traditional bench synthesis. The solid support can be, for example, chlorotrityl (Cl) or Wang (OH) resin, both of which are readily available commercially. The active groups of those resins react readily with the carboxyl group of an N-Fmoc amino acid, thereby covalently binding it to the polymer. The resin-bound amine may be deprotected by exposure to piperidine. A second N-protected amino acid may then be coupled to the resin-amino acid. These steps are repeated until the desired sequence is obtained. At the end of the synthesis, the resin-bound protected peptide may be deprotected and cleaved from the resin with trifluoroacetic acid (TFA). Examples of reagents facilitating the coupling new amino acids to the resin-bound amino acid chain are: tetra-methyluronium hexafluorophosphate (HATU), O-(1H-benzotriazole-1-yl)-N,N,N′,N′-tetramethyluronium hexafluorophosphate (HBTU), O-(1H-benzotriazole-1-yl)-N,N,N′,N′-tetramethyluronium tetrafluoroborate (TBTU), 1H-hydroxybenzotriazole (HOBt).

Peptide synthesis by solution chemistry rather than solid phase chemistry is also feasible.

Modification of a side-chain amino or carboxyl group of an amino acid in the peptide chain, for example to introduce a GAG-binding or other motif as described above, is a simple matter of selective protection and deprotection of other reactive side-chain groups not to be involved in the reaction.

### EXAMPLE

To illustrate methods which are applicable to the synthesis of agonists of the invention, the preparation of the sequence [Gln34]PP will be described.

Overview of synthesis:

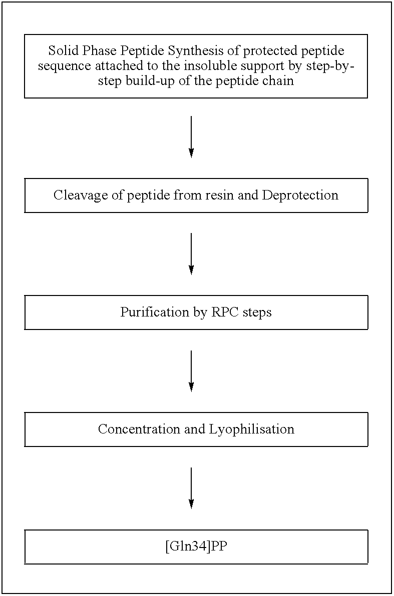

### Chemicals

The following starting materials and solvents were used:

### Starting Material

### Fmoc-Rink-TentaGel-resin

### Fmoc-Ala-OH.H₂O

### Fmoc-Arg(Pbf)-OH

### Fmoc-Asn(Trt)-OH

### Fmoc-Asp(OtBu)-OH

### Fmoc-Gln(Trt)-OH

### Fmoc-Glu(OtBu)-OH

### Fmoc-Gly-OH

### Fmoc-Ile-OH

### Fmoc-Leu-OH

### Fmoc-Met-OH

### Fmoc-Pro-OH

### Fmoc-Thr(tBu)-OH

### Fmoc-Tyr(tBu)-OH

### Fmoc-Val-OH

SOLVENTS AND REAGENTS USED IN THE SYNTHESIS AND
FOR THE PURIFICATION

| | |
|---|---|
| Acetic acid | Solvent |
| Acetic anhydride | Reagent |
| Ammonium acetate | Reagent |
| Ammonium iodide | Reagent |
| Diisopropylcarbodiimide (DIC) | Reagent |
| Dimethylformamide (DMF) | Solvent |
| Dithiothreitol (DTT) | Reagent |
| Ethanol | Solvent |
| 1-Hydroxybenzotriazole (HOBt) | Reagent |
| Isopropanol | Solvent |
| N-Methylmorpholine (NMM) | Reagent |
| Piperidine | Reagent |
| Trifluoroacetic acid (TFA) | Solvent |

The required product was assembled by Fmoc-SPPS on a Rink-TentaGel-resin. The amino acids having side chain protection were: Arg(Pbf), Asn(Trt), Asp(OtBu), Gln(Trt), Glu(OtBu), Thr(tBu) and Tyr(tBu).

The couplings were performed in DMF with variable amino acid equivalents using DIC and HOBt for activation. Each coupling was followed by capping using acetic anhydride and NMM. Monitoring of each coupling was carried out using either a Kaiser/ninhydrin or chloranil test. Between each coupling cycle the Fmoc group was removed with piperidine in DMF.

The protected peptide-resin was cleaved from the support by TFA, and neutralised in ammonium acetate in water, producing the crude product. After treatment, the resin was removed by filtration.

The crude product was purified by preparative HPLC using silica based or polystyrene based reverse phase materials. The peptide was then desalted by reverse phase chromatography. The peptide solution was concentrated by evaporation and filtered before isolation by lyophilisation.

The structure was confirmed by Collision Induced Dissociation Mass Spectrometry/Mass Spectrometry (CID MS/MS), amino acid analysis, LC-MS, and chiral purity. The purity was assessed by HPLC, chiral purity.

### Chemical Analysis of Peptides:

Analytical data for some of the peptides of the invention, synthesised by the above methods, are set out below, by way of illustration of the methods used and results achieved:

Data

| | | | | Measured | | | |
|---|---|---|---|---|---|---|---|
| | Molecular | | Theoretical | Mass | Rt | | HPLC |
| Peptide | formula | MW | m/z | M/z | min | Purity | Method |
| [Gln34]PP | C185H288N54O55S2 | 4212.8 | | 4212 | 20.8 | 94.8 | C |
| Ile31,Gln34]PP | C185H288N54O55S2 | 4212.8 | | 4211.6 | 20.5 | 97.1 | C |
| [Val31,Gln34]PP | C184H286N54O55S2 | 4198.8 | | 4197.2 | 25.0 | 96.3 | C |
| Leu30,Gln34]PP | C186N289N53O56S | 4195.8 | 840.2 [M + 5H] | 840.0 [M + 5H] | 19.7 | 96.5 | B |
| His26,Gln34]PP | C185H282N52O56S2 | 4194.8 | 839.9 [M + 5H] | 839.9 [M + 5H] | 19.1 | 95.0 | B |
| [Ala1,Glu4,Arg26, | C186H288N54O59S | 4256.8 | 710.4 [M + 6H] | 710.4 [M + 6H] | 19.4 | 96.4 | B |
| Met30]PYY | | | | | | | |
| [Arg26,Met30]PYY | C193H298N56O57S1 | 4346.9 | 870.7 [M + 5H] | 870.7 [M + 5H] | 20.2 | 91.3 | B |
| Glu4,Arg26]PYY | C193H295N55O59 | 4329.8 | 867.5 [M + 5H] | 867.5 [M + 5H] | 17.4 | 95.3 | B |

### Analytical HPLC Method A

### Column=Vydac C18 Peptide-Protein Column, 250×4.6 mm

Buffer A=0.05% TFA in water

### Buffer B=0.05% TFA in 100% MeCN

### Gradient=0% B to 60% B in 20 min

Flow rate=1.00 mL/min

### Wavelength=215 nm

Mass spectroscopy=MALDI-TOF with gentisic acid or αcyanohydroxy cinnamic acid as matrix.

### Analytical HPLC Method B

### Column=Hypersil ODS-2, 250×4.6 mm

### Buffer A=0.1% TFA in 100% MeCN

Buffer B=0.1% TFA in 100% water

### Gradient=24% A to 35% A in 25 min

Flow rate=1.00 mL/min

### Wavelength=220 nm

Mass spectroscopy=ESI [nebuliser gas flow: 1.5 L/min; CDL −20.0 v; CDL temp: 250° C.; Block temp: 200° C.; probe bias: +4.5 kv; Detector: 1.5 kv; T. Flow: 0.2 mL/min; B. conc: 50% H20/50% CAN.]

### Analytical HPLC Method C

### Column=Vydac C18 218TP54, 250×4.6 mm

Buffer A=20 mL of MeCN and 2 mL of TFA in Water (total volume 2000 mL)
Buffer B=2 mL of TFA in Water (total volume 2000 mL)
Gradient=25% B to 75% B over 27 min
Flow rate=1.00 mL/min

### Wavelength=215 nm

Injection volume=10 μL

As further illustration of the synthetic methods which may be used for the preparation of the Y2/Y4 selective agonists with which the invention is concerned, the following protocols are set out, by way of example only:

Synthesis of [Cys2,Lys13,D-Cys27,Gln34]PP (SEQ ID No: 30) and analogs thereof

In the following, a synthesis of the cyclic Y2 selective peptide [Cys2,Lys13,D-Cys27,Gln34]PP (SEQ ID No: 30), and the synthesis of analogs with either a GAG-binding motif, a serum protein binding motif or a polyethyleneglycol moiety attached at the epsilon amino group of Lys13, is described.

In general side group protection is standard Fmoc except for:

### Arg=Fmoc Arg(Pbf)-OH

### Asn, Gln=Fmoc Asn(Trt)-OH

Thr, Ser, Asp, Glu, Tyr=tButyl

### Lys=Fmoc Lys(tBoc)-OH

Ala-Ser 22-23=Fmoc AlaSer pseudoproline

In the case of [Cys2,Lys13,D-Cys27,Gln34]PP the following special protection groups were used in order to obtain selective deprotection:

### Lys13=Fmoc Lys(Dde)-OH

### Cys 27=Fmoc DLys(Trt)-OH

The peptide is synthesized by solid phase synthesis, on PAL Peg-PS resin (a resin which will generate the biologically important carboxyamide group upon cleavage), using Fmoc chemistry with a 5 fold molar reagent excess. The coupling is performed by HCTU throughout using DMF as solvent. Fmoc removal after each coupling step is performed with 20% piperidine in DMF for 10-15 minutes. The coupling is checked after each step by quantitative ninhydrin assay. In certain cases double couplings could be performed.

After the synthesis of the full length peptides, the protection group on the epsilon amino group of Lys13 is selectively removed by treatment with 2% hydrazine in DMF for 15-20 mins, while the peptide is still attached to the resin.

The resin is subsequently divided into different batches to generate the underivatized peptide as well as three different motif-modified peptides:

1. [Cys2,Lys13,D-Cys27,Gln34]PP (SEQ ID No: 30)—the “mother peptide”—is cleaved from the resin and deprotected by treatment with 95% trifluoroacetic acid: 2.5% water: 2.5% tripropyl silane for 2-3 hours.

The intramolecular stabilizing disulfide bridge between Cys2 and D-Cys27 is generated by air oxidation by dissolving the peptide at <1 mg/ml, in ammonium acetate pH 8.5-9 and stirring for 24-48 hours until no free thiols can be detected by the Ellman assay.

The peptide is purified by reverse phase HPLC: Vydac 300 Å column, 250 mm×10 mm column eluted with (Buffer A=0.05% TFA in water; Buffer B=60% MeCN: 40% water: 0.05% TFA) in a gradient of 30-80% buffer B over 20 mins, 2 ml/min. OD 215 nM was measured and the eluant containing the specific peptide is collected and lyophilized.

The structure of the peptide is confirmed by mass spec, amino acid analysis and if desired also by amino acid sequence analysis.

2. [Cys2, N-(8-(8-gammaglutamoylamino-octanoylamino)-octanoyl)-Lys13, D-Cys27,Gln34]PP (SEQ ID No: 31)—In order to attach a serum albumin binding motif, an 8-(8-gammaglutamoylamino-octanoylamino)-octanoyl group is linked to the free epsilon amino group of Lys13 after removal of the protecting Dde group while the peptide is still attached to the resin by peptide synthesis using protected aminooctanoic acid twice followed by protected gammaglutamic acid.

The motif-modified peptide is cleaved from the resin, deprotected, cyclized and purified as described above for the “mother peptides”.

3. Fluorescein-[Cys2, N-{(Ala-Arg-Arg-Arg-Ala-Ala-Arg-Ala)₃}-Lys13,D-Cys27,Gln34]PP (SEQ ID No: 32)—In order to attach a GAG-binding motif, the motif is built stepwise by peptide synthesis using the free epsilon amino group of Lys13, after removal of the Dde group, on the [Cys2,Lys13,D-Cys27,Gln34]PP peptide still attached to the resin, using standard Fmoc chemistry as described above in general: The GAG binding sequence attached in this way is Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala. A fluorescein tag group is added for in vitro and in vivo analytical purposes as its 5,6 isomer —NHS ester, 10× excess over 72 hours—to the N-terminus of the final GAG-binding sequence.

The GAG binding-motif modified peptide is cleaved from the resin, deprotected, cyclized and purified as described above for the “mother peptide”.

4. [Cys2, N-{N′-(21-amino-4,7,10,13,16,19-hexaoxaheneicosanoyl)}-gammaglutamoyl-Lys13,D-Cys27,Gln34]PP (SEQ ID No: 33)—In order to attach a polyethylenglycol moiety to the Y2-Y4 selective peptide, protected 21-amino-4,7,10,13,16,19-hexaoxaheneicosanoic-acid is joined by peptide synthesis using the free epsilon amino group of Lys13 after removal of the Dde grup on the [Cys2,Lys13,D-Cys27,Gln34]PP peptide still attached to the resin, followed by a protected gamma-glutamic acid.

The PEGylated peptide is cleaved from the resin, deprotected, cyclized and purified as described above for the “mother peptide”.

Using the above methods, the following agonists of the invention are synthesized as examples:

[N—(N′-hexadecanoyl)-gammagluatamoyl-Lys13,Gln34]PP (SEQ ID No: 34)

### [N-(Ala-Arg-Arg-Arg-Ala-Ala-Arg-Ala)₃-Lys18,Gln34]PP (SEQ ID No: 35)

### [N-PEG5000-Lys13,Gln34]PP (SEQ ID No: 36)

### [Ile31,Gln34]PP (SEQ ID No: 5)

### [Val31,Gln34]PP (SEQ ID No: 6)

### [Leu30,Gln34]PP (SEQ ID No: 7)

### [Nle30,Gln34]PP (SEQ ID No: 8)

### [Leu28,Gln34]PP (SEQ ID No: 9)

### [His26,Gln34]PP (SEQ ID No: 10)

### [Ile3,Gln34]PP (SEQ ID No: 11)

### [Ala1,Glu4,Arg26,(Met30 or Nle30)]PYY (SEQ ID No: 12)

[Ala1,Glu4, N—(N′-hexadecanoyl)-gammagluatamoyl-Lys13,Arg26,Nle30]PYY (SEQ ID No: 37)

### [Ala1,Glu4, N-(Ala-Arg-Arg-Arg-Ala-Ala-Arg-Ala)₃-Lys13,Arg26, Nle30]PYY (SEQ ID No: 38)

### [Ala1,Glu4,N-PEG5000-Lys13,Arg26,Nle30]PYY (SEQ ID No: 39)

### [Ala1,Glu4,Arg26(Met30 or Nle30)]NPY (SEQ ID No: 13)

### [Ala1,Glu4]PYY (SEQ ID No: 14) and [Ala1,Glu4]NPY (SEQ ID No: 15)

### [Arg26, (Met30 or Nle30)]PYY (SEQ ID No: 16) and [Arg26, (Met30 or Nle30)]NPY (SEQ ID No: 17)

### [Glu4, (Met30 or Nle30)]PYY (SEQ ID No: 18) and [Glu4, (Met30 or Nle30)]NPY (SEQ ID No: 19)

### [Glu4,Arg26]PYY (SEQ ID No: 20) and [Glu4,Arg26]NPY (SEQ ID No: 21)

### [Cys2,D-Cys27,Gln34]PP (SEQ ID No: 22)

### [Cys2,Aoc5-24,D-Cys27,Gln34]PP (SEQ ID No: 23)

### Biological Assays and Results

### I. In Vitro Assays to Determine Peptide Affinity and Potency

### Human Y2 Receptor Affinity Assay

Affinity of test compounds for the human Y2 receptor is determined in a competition binding assay using 125I-PYY binding in COS-7 cells transiently transfected with the human Y2 receptor using a standard calcium phosphate transfection method.

Transfected COS-7 cells are transferred to culture plates one day after transfection at a density of 40×103 cells per well aiming at 5-8% binding of the radioactive ligand. Two days after transfection, competition binding experiments are performed for 3 hours at 4 C.° using 25 pM of 125I-PYY (Amersham, Little Chalfont, UK). Binding assays are performed in 0.5 ml of a 50 mM Hepes buffer, pH 7.4, supplemented with 1 mM CaCl2, 5 mM MgCl2, and 0.1% (w/v) bovine serum albumin and 100 μg/ml bacitracin. Non-specific binding is determined as the binding in the presence of 1 μM of unlabeled PYY. Cells are washed twice in 0.5 ml of ice-cold buffer and 0.5-1 ml of lysis buffer (8 M Urea, 2% NP40 in 3 M acetic acid) is added and the bound radioactivity is counted in a gamma counter. Determinations are made in duplicate. Steady state binding is reached with the radioactive ligand under these conditions. EC50 values were calculated using a standard pharmacological data handling software, Prism 3.0 (graphPad Sofware, San Diego, USA).

### Human Y4 Receptor Affinity Assay

Protocol as for the Y2 affinity assay, except that human Y4-transformed COS-7 cells are used, the competition assay uses 125I-PP, and PP is used for the determination of non-specific binding.

### Human Y1 Receptor Affinity Assay

Protocol as for the Y2 affinity assay, except that human Y1-transformed COS-7 cells are used and are transferred to culture plates at a density of 1.5×106 cells per well. the competition assay uses 125I-NPY, and NPY is used for the determination of non-specific binding.

The results of testing NPY, PYY. PYY3-36, PP and the [Gln45]PP, [Ile31,Gln34]PP, [Val31,Gln34]PP, [Arg26,Met30]PYY, and [Glu4,Arg26]PYY agonists of the invention in the above affinity assays are given in Table 1:

TABLE 1

| | Y2 | | | Y4 | | | Y1 | | |
|---|---|---|---|---|---|---|---|---|---|
| | IC50 | | | IC50 | | | IC50 | | |
| Agonist | nm | SEM | n | nm | SEM | n | nm | SEM | n |
| NPY | 0.30 | 0.07 | 4 | 26 | 5. | 4 | 2.3 | 0.3 | 4 |
| PYY | 0.22 | 0.02 | 2 | 30 | 8.0 | 2 | 16 | 1 | 2 |
| PYY(3-36) | 0.20 | 0.03 | 10 | 343 | 42 | 4 | >1000 | | 2 |
| PP | >1000 | | 4 | 0.41 | 0.05 | 11 | >1000 | | 1 |
| [Gln34]-PP | 9.0 | 1.8 | 3 | 1.2 | 0.1 | 4 | >1000 | | 2 |
| Ile31,Gln34]-PP | 54 | 22 | 3 | 1.1 | 0.1 | 3 | >1000 | | 2 |
| [Val31,Gln34]-PP | 69 | 20 | 3 | 0.93 | 0.16 | 3 | >1000 | | 2 |
| [Arg26,Met30]PYY | 0.13 | | 1 | 2.3 | | 1 | 65 | | 1 |
| [Glu4,Arg26]PYY | | | | 3.3 | | 1 | 367 | | 1 |

### Human Y2 Receptor Potency Assay

Potency of the test compounds on the human Y2 receptor is determined by performing dose-response experiments in COS-7 cells transiently transfected with the human Y2 receptor as well as a promiscuous G protein, Gqi5 which ensures that the Y2 receptor couples through a Gq pathway leading to an increase in inositol phosphate turnover.

Phosphatidylinositol turnover—One day after transfection COS-7 cells are incubated for 24 hours with 5 μCi of [3H]-myo-inositol (Amersham, PT6-271) in 1 ml medium supplemented with 10% fetal calf serum, 2 mM glutamine and 0.01 mg/ml gentamicin per well. Cells are washed twice in buffer, 20 mM HEPES, pH 7.4, supplemented with 140 mM NaCl, 5 mM KCl, 1 mM MgSO4, 1 mM CaCl2, 10 mM glucose, 0.05% (w/v) bovine serum; and are incubated in 0.5 ml buffer supplemented with 10 mM LiCl at 37 C for 30 min. After stimulation with various concentrations of peptide for 45 min at 37 C, cells are extracted with 10% ice-cold perchloric acid followed by incubation on ice for 30 min. The resulting supernatants are neutralized with KOH in HEPES buffer, and the generated [3H]-inositol phosphate are purified on Bio-Rad AG 1-X8 anion-exchange resin and counted in a beta counter. Determinations are made in duplicates. EC50 values were calculated using a standard pharmacological data handling software, Prism 3.0 (graphPad Sofware, San Diego, USA).

### Human Y4 Receptor Potency Assay

Protocol as for the Y2 potency assay, except that human Y4-transformed COS-7 cells are used.

### Human Y1 Receptor Potency Assay

Protocol as for the Y2 potency assay, except that human Y1-transformed COS-7 cells are used.

The results of testing NPY, PYY, PYY3-36, PP and [Gln45]PP, [Ile31,Gln34]PP, [Val31,Gln34]PP (SEQ ID No: 6), [Ala1,Glu4,Arg26,Met30]PYY, [Leu30,Gln34]PP (SEQ ID No: 7) and [His26,Gln34]PP (SEQ ID No: 10) of the agonists of the invention in the above potency assays are given in Table 2:

TABLE 2

| | Y2 | | | Y4 | | | Y1 | | |
|---|---|---|---|---|---|---|---|---|---|
| | EC50 | | | EC50 | | | EC50 | | |
| Agonist | nm | SEM | N | nm | SEM | n | nm | SEM | n |
| NPY | 1.5 | 0.5 | 11 | 167 | 56 | 7 | 1.7 | 0.4 | 11 |
| PYY | 0.23 | 0.06 | 8 | 34 | 5 | 6 | 0.6 | 0.1 | 5 |
| PYY(3-36) | 0.36 | 0.07 | 16 | >1000 | | 8 | 74 | 5 | 7 |
| PP | | | 8 | 0.64 | 0.07 | 17 | 83 | 13 | 5 |
| [Gln34]-PP | 3.5 | 0.7 | 5 | 1.2 | 0.1 | 5 | 388 | 95 | 3 |
| Ile31,Gln34]-PP | 6.5 | 2.0 | 2 | 0.91 | 0.04 | 3 | 48 | 9 | 3 |
| [Val31,Gln34]-PP | 9.9 | 3.2 | 2 | 1.0 | 0.04 | 2 | 99 | 14 | 3 |
| [Leu30,Gln34]PP | 8.6 | 0.7 | 3 | 4.5 | 0.4 | 3 | 102 | 7 | 4 |
| [His26,Gln34]PP | 6.1 | 1.1 | 3 | 9.2 | 0.3 | 3 | 130 | 23 | 4 |
| [Ala1,Glu4,Arg26,Met30]PYY | 1.0 | 0.2 | 3 | 1.4 | 0.2 | 3 | 87 | 16 | 3 |

### II. In Vitro Assays to Determine Protein Stability

An important measure for many of the peptides of the invention is the protein stability especially in respect of stability for example towards degradation by enzymes as the peptides have been designed to have increased stability as compared to for example PYY3-36 or even increased stability as compared to full length PYY and PP.

Stability of the PP-fold—is determined as the stability of the peptides towards degradation by endopeptidases which cleave for example in the loop region, which is relatively flexible as described (O'Hare, M. & Schwartz, T. W 1990 In Degradation of Bioactive Substances Physiology and Pathophysiology. J. Henriksen, ed. CRC Press, Boca Raton, Fl.). As a model enzyme, endoproteinase Asp-N (Pierce) is used. This enzyme cleave at the N-terminal side of Asp residues, for example between residues 9 and 10 (Asp) in PP. The peptides are incubated with an efficacious dose of endopeptidase Asp-N as indicated by the manufacturer in 0.01 M Tris/HCl buffer, pH 7.5 at room temperature and samples removed after various time periods over 24 hours. The samples are analysed by HPLC and the gradual degradation of the peptides is followed over time. The peptides are compared in respect of stability to PYY, PYY13-36 and PP.

Stability towards aminopeptidases—is determined as described above for endopeptidases but using aminopeptidase N and di-peptidylpeptidase IV in stead. Some of the peptides are specially designed to be resistant to these aminopeptidase, for example PYY2-36, the N-terminally acetylated peptide derivates, and peptide derivates alkylated with an albumin binding moiety at the N-terminus. The peptides are compared in respect of stability to PYY, PYY3-36 and PP.

### III. In Vitro Assay to Determine Binding to Glycosamino Glycans (GAGS)

The ability of test compounds to bind to GAGs is monitored in an in vitro assay using immobilized heparin, i.e. a heparin agarose as affinity matrix. using either HiTrap heparin-Sepharose column (Amersham Pharmacia Biotech, Uppsala, Sweden) or heparin HPLC columns which are eluted with a 50-min linear gradient of 0-0.5 M NaCl in 50 mM sodium phosphate (pH 7.3) containing 2 mM DTT and 1 mM MgEDTA at a flow rate of 1 ml/min. For regeneration, the column was washed with 1 M NaCl in buffer A from 51-55 min.

### IV. In Vivo Studies to Determine the Effect of the Peptides on Appetite, Food Intake and Body Weight

### Effect of Y2/Y4 Over Y1 Selective Agonists on Acute Food Intake in Mice

The ddy strain of mice were used, 34-37 g and 8-9 weeks of age (Japan SLC, Shizuuoka, Japan). The mice were individually house in a regulated environment (22 C, 55% humidity) in a 12-hour light-dark cycle with light on at 7 AM. Food and water were available ad libitum except just before experiments (see below). The mice were acclimatized to subcutaneous injections during the week before experiments started. Mice were used once each in the experiments. Just before administration the peptides were diluted in physiological saline and administered in 100 μL volume for subcutaneous administration. Results are expressed as mean +/−SE. Analysis of variance followed by Bonferroni's test were used to assess differences among groups.

Mice were deprived of food but with free access to water for 16 hours prior to the actual test and experiments were started at 10 AM on the following day. A standard diet was used (CLEA Japan, Inc., Tokyo, Japan) and food intake was measured by subtracting uneaten food from the initially premeasured food after administration and checking for food spillage. Eight animals in each group receiving either saline, 3 μg PYY3-36, 30 μg PYY3-36, 10 μg test compound, or 100 μg test compound.

The results for [Gln34]PP (called TM30333 in the Fig) as test compound are shown in FIG. 2.

1. A composition for treatment of conditions responsive to activation of Y2 and/or Y4 receptors comprising a Y receptor agonist which is selective for the Y2 and Y4 receptors over the Y1 receptor, and a suitable excipient
(a) the said agonist being a PP-fold peptide or PP-fold peptide mimic which has
(i) a C-terminal Y receptor-recognition amino acid sequence represented by —X-Thr-Arg-X³-Arg-Tyr-C(═O)NR¹R² wherein R¹ and R² are independently hydrogen or C₁-C₆ alkyl X is Val, Ile, Leu or Ala, and X³ is Gln or Asn, or a conservatively substituted variant thereof in which Thr is replaced by His or Asn and/or Tyr is replaced by Trp or Phe; and/or Arg is replaced by Lys, and
(ii) an N-terminal Y receptor-recognition amino acid sequence represented by H₂N—X¹-Pro-X²—(Glu or Asp)—wherein X¹ is not present or is any amino acid residue, and X² is Leu or Ser or conservative substitutions of Leu or Ser, or
(b) the said agonist comprising
a C-terminal Y receptor-recognition amino acid sequence as defined in (i) above,
said Y receptor-recognition sequence being fused to an amphiphilic amino acid sequence domain comprising at least one alpha helical turn adjacent the N-terminus of the said hexapeptide sequence,
said turn being constrained in a helical configuration by a covalent intramolecular link, and optionally
an N-terminal sequence which commences with a Y receptor-recognition amino acid sequence as defined in (ii) above.

2. The composition as claimed in claim 1 wherein, in the C terminal Y receptor-recognition amino acid sequence of the agonist, R¹ and R² are each hydrogen.

3. The composition as claimed in claim 1 wherein, in the C-terminal Y receptor-recognition amino acid sequence of the agonist, residue X is Val, or Ile.

4. The composition as claimed in claim 1 wherein the agonist comprises a C-terminal Y receptor-recognition sequence represented by —X^{A}—X-Thr-Arg-X³-Arg-TyrC(═O)NR¹R² wherein the residue X^{A} is non-basic and non-acidic, and the sequence —X-Thr-Arg-X³-Arg-Tyr-C(═O)NR¹R² is as defined in claim 1.

5. The composition as claimed in claim 4 wherein, in the C-terminal Y receptor-recognition sequence of the agonist, the said non-basic and non-acidic amino acid residue is Leu, Met, Ile, Val, or Ala.

6. The composition as claimed in claim 1 wherein the agonist comprises a C-terminal undecapeptide represented by —X^{c}-Tyr-X^{B}-Asn-X^{A}—X-Thr-Arg-X³-Arg-Tyr-C(═O)NR¹R² wherein the sequence —X^{A}—X-Thr-Arg-X³-Arg-Tyr-C(═O)NR¹R² is as defined in claim 4, X^{C} is Arg or Lys and X^{B} is Ile, Leu or Val.

7. The composition as claimed in claim 1 wherein the agonist comprises a C-terminal undecapeptide sequence represented by —X^{C}-Tyr-X^{B}-Asn-X^{A}—X-Thr-Arg-X³-Arg-Tyr-C(═O)NR¹R² wherein the sequence —X^{A}—X-Thr-Arg-X³-Arg-Tyr-C(═O)NR¹R² is as defined in claim 4, X^{C} is His, Asn, or Gln and X^{B} is Ile, Leu or Val.

8. The composition as claimed in claim 1 wherein, in the C-terminal Y receptor-recognition amino acid sequence of the agonist, X³ is Gln.

9. The composition as claimed in claim 1 wherein the agonist comprises the C-terminal undecapeptide sequence -Arg-Tyr-Leu-Asn-(Leu or Met)-Val-Thr-Arg-Gln-Arg-Tyr-C(═O)NH₂.

10. The composition as claimed in claim 1 wherein the agonist comprises the C-terminal undecapeptide sequence -His-Tyr-(Ile or Leu)-Asn-Met-Leu-Thr-Arg-Gln-Arg-Tyr-C(═O)NH₂.

11. The composition as claimed in claim 1 wherein, in the N-terminal Y receptor-recognition amino acid sequence of the agonist, when present, the residue X¹ is Ala, or is absent.

12. The composition as claimed in claim 1 wherein, in the N-terminal Y receptor-recognition amino acid sequence of the agonist, when present, the residue X² is Leu, Ile, or Ser.

13. The composition as claimed in claim 1 wherein the N-terminal sequence is H₂N-Ala-Pro-Leu-Glu-, or H₂N-Pro-Leu-Glu-.

14. The composition as claimed in claim 1 wherein the agonist is of type (b), with an N-terminal Y receptor-recognition sequence, and has a PP-fold structure in which the helical turn-constraining intramolecular link extends from an amino acid residue in the amphiphilic domain to a linkage point in the N-terminal part of the agonist corresponding to the polyproline domain of a PP-fold peptide which extends antiparallel to the amphiphilic domain.

15. The composition as claimed in claim 14 wherein, in the agonist, the helical turn-constraining intramolecular link is a disulfide or lactam link.

16. The composition as claimed in claim 15 wherein in the agonist the covalent intramolecular link in the agonist is a disulfide link formed between an L- or D-Cys residue in the alpha helix and a Cys residue located in the N-terminal part of the agonist corresponding to the polyproline domain of a PP-fold peptide which extends antiparallel to the amphiphilic domain.

17. The composition as claimed in claim 1 wherein the agonist is of type (b), and in the agonist the helical turn-constraining. intramolecular link is a lactam link formed between Lys and Glu residues in the said helical turn, or between a Lys or Glu residue in the said helical turn and a Glu or Lys residue in the C-terminal Y receptor recognition sequence.

18. The composition as claimed in claim 1 wherein the agonist has both a C-terminal and N-terminal Y receptor recognition sequence, the C-terminal sequence being fused at its N-terminus to an amphiphilic amino acid sequence domain comprising at least one alpha helical turn adjacent the N-terminus of the said epitope, the said C- and N-terminal amino acid sequences being joined by peptide bonds to the carboxyl and amino groups respectively of an amino acid of formula NH₂(CH₂)ₙCO₂H wherein n is from 2 to 12.

19. The composition as claimed in claim 18 wherein, in the agonist, n is 6, 7, 8, 9 or 10.

20. The composition as claimed in claim 1 wherein the agonist is selected from
[Gln34] PP (SEQ ID No:4)
[N—(N′-hexadecanoyl)-gammagluatamoyl-Lys13,Gln34]PP (SEQ ID No: 34)
[N-(Ala-Arg-Arg-Arg-Ala-Ala-Arg-Ala)₃-Lys18,Gln34]PP (SEQ ID No: 35)
[N-PEG5000-Lys13,Gln34]PP (SEQ ID No: 36)
[Ile31,Gln34]PP (SEQ ID No: 5)
[Val31,Gln34]PP (SEQ ID No: 6)
[Leu30,Gln34]PP (SEQ ID No: 7)
[Nle30,Gln34]PP (SEQ ID No: 8)
[Leu28,Gln34]PP (SEQ ID No: 9)
[His26,Gln34]PP (SEQ ID No: 10)
[Ile3,Gln34]PP (SEQ ID No: 11)
[Ala1,Glu4,Arg26,(Met30 or Nle30)]PYY (SEQ ID No: 12)
[Ala1,Glu4,N—(N′-hexadecanoyl)-gammagluatamoyl-Lys13,Arg26,Nle30]PYY (SEQ ID No: 37)
[Ala1,Glu4,N-(Ala-Arg-Arg-Arg-Ala-Ala-Arg-Ala)₃-Lys13,Arg26,Nle30]PYY (SEQ ID No: 38)
[Ala1.Glu4,N-PEG5000-Lys13,Arg26,Nle30]PYY (SEQ ID No: 39)
[Ala1,Glu4,Arg26(Met30 or Nle30)]NPY (SEQ ID No: 13)
[Ala1,Glu4]PYY (SEQ ID No: 14) and [Ala1,Glu4]NPY (SEQ ID No: 15)
[Arg26, (Met30 or Nle30)]PYY (SEQ ID No: 16) and [Arg26, (Met30 or Nle30)]NPY (SEQ ID No: 17)
[Glu4, (Met30 or Nle30)]PYY (SEQ ID No. 18) and [Glu4, (Met30 or Nle30)]NPY (SEQ ID No: 19)
[Glu4,Arg26]PYY (SEQ ID No: 20) and [Glu4,Arg26]NPY (SEQ ID No: 21)
[Cys2,D-Cys27,Gln34]PP (SEQ ID No: 22)
[Cys2,Aoc5-24,D-Cys27,Gln34]PP (SEQ ID No: 23)
[Cys2,Lys13,D-Cys27,Gln34]PP (SEQ ID No: 30)
[Cys2,N-(8-(8-gammaglutamoylamino-octanoylamino)-octanoyl)-Lys13,D-Cys27,Gln34]PP (SEQ ID No: 31)
[Cys2, N-{(Ala-Arg-Arg-Arg-Ala-Ala-Arg-Ala)₃}-Lys13,D-Cys27,Gln34]PP (SEQ ID No: 32)
[Cys2,N-{N′-(21-amino-4,7,10,13,16,19-hexaoxaheneicosanoyl)}-gammaglutamoyl-Lys13,D-Cys27,Gln34]PP (SEQ ID No: 33)
and conservatively substituted analogues thereof.

21. The composition as claimed in claim 1 wherein the agonist is [Gln34]PP (SEQ ID No:4) or [Ala1,Glu4,Arg26, (Met30 or Nle30)]PYY (SEQ ID No: 12) or a conservatively substituted analogue thereof.

22. The composition as claimed in claim 1 wherein the agonist is acylated at its N-terminus to confer resistance to aminopeptidase activity.

23. The composition as claimed in claim 22 wherein the agonist is acylated at its N-terminus with a carbon chain having from 2 to 24 carbon atoms.

24. The composition as claimed in claim 22 wherein the agonist is acetylated at its N-terminus.

25. The composition as claimed in claim 1 wherein the agonist is as defined in any such claim, and comprises a serum albumin binding motif, or a glycosaminoglycan (GAG) binding motif, or a helix inducing motif, or is PEGylated.

26. The composition as claimed in claim 25 wherein, in the agonist, the serum albumin binding motif is a lipophilic group.

27. The composition as claimed in claim 26 wherein, in the agonist, the lipophilic group comprises an optionally substituted, saturated or unsaturated, straight or branched hydrocarbon group of from 10 to 24 carbon atoms.

28. The composition as claimed in claim 25, wherein, in the agonist, the lipophilic group is, or is part of, a side chain to the backbone of the agonist.

29. The composition as claimed in claim 28 wherein, in the agonist, the lipophilic group-containing side chain is connected to a residue in the backbone via an ether, thioether, amino, ester or amide bond.

30. The composition as claimed in claim 29 wherein, in the agonist, the lipophilic group-containing side chain is selected from the group consisting of:
CH₃(CH₂)ₙCH(COOH)NH—CO(CH₂)₂CONH— wherein n is an integer from 9 to 15,
CH₃(CH₂)ᵣCO—NHCH(COOH)(CH₂)₂CONH— wherein r is an integer form 9 to 15, and
CH₃(CH₂)ₛCO—NHC((CH₂)₂COOH)CONH— wherein s is an integer from 9 to 15,
CH₃(CH₂)ₘCONH—, wherein m is an integer from 8 to 18,
—NHCOCH((CH₂)₂COOH)NH—CO(CH₂)ₚCH₃, wherein p is an integer from 10 to 16, and
—NHCO(CH₂)₂CH(COOH)NH—CO(CH₂)_{q}CH₃, wherein q is an integer from 10 to 16,
CH₃(CH₂)ₙCH(COOH)NHCO—, wherein n is an integer from 9 to 15, CH₃(CH₂)ₚNHCO—, wherein p is an integer from 10 to 18—CONHCH(COOH)(CH₂)₄NH—CO(CH₂)ₘCH₃, wherein m is an integer from 8 to 18, —CONHCH(COOH)(CH₂)₄NH—COCH((CH₂)₂COOH)NH—CO(CH₂)ₚCH₃, wherein p is an integer from 10 to 16,
—CONHCH(COOH)(CH₂)₄NH—CO(CH₂)₂CH(COOH)NH—CO(CH₂)_{q}CH₃, wherein q is an integer from 10 to 16, and
a partly or completely hydrogenated cyclopentanophenanthrene skeleton.

31. The composition as claimed in claim 28 wherein, in the agonist, the lipophilic group-containing side chain is a C₁₂, C₁₄, C₁₆ or C₁₈ acyl group acylating an amino group present in the side chain of a residue of the backbone of the agonist.

32. The composition as claimed in claim 28 wherein, in the agonist, the lipophilic group-containing side chain is a tetradecanoyl group acylating an amino group present in the side chain of a residue of the backbone of the agonist.

33. The composition as claimed in claim 1 wherein the agonist is Lys11-tetradecanoyl-[Lys11,Gln34]PP,[N′-hexadecanoyl)-gammagluatamoyl-Lys13,Gln34]PP (SEQ ID No: 34], [Ala1,Glu4,N—(N′-hexadecanoyl)-gammagluatamoyl-Lys13,Arg26,Nle30]PYY (SEQ ID No. 37), or [Cys2,N-(8-(8-gammaglutamoylamino-octanoylamino)-octanoyl)-Lys13,D-Cys27,Gln34]PP (SEQ ID No: 31,
or a conservatively substituted analogue thereof.

34. The composition as claimed in claim 25, wherein, in the agonist, the GAG binding motif is an amino acid sequence which is, or is part of, a side chain to the backbone of the agonist.

35. The composition as claimed in claim 34 wherein, in the agonist, the GAG-binding motif comprises the amino acid sequence XBBXBX and/or XBBBXXBX, wherein B is a basic amino acid residue and X is any amino acid residue.

36. The composition as claimed in claim 34 wherein, in the agonist, the GAG-binding motif is concatameric or dendrimeric.

37. The composition as claimed in claim 34 wherein the GAG-binding motif is Ala-Arg-Arg-Arg-Ala-Ala-Arg-Ala-Ala-Arg-Arg-Arg-Ala-Ala-Arg-Ala coupled through an amide bond formed between the C-terminus of the concatameric GAG-binding motif and the epsilon amino group of Lys18 in [Lys18,Gln34]PP (SEQ ID No: 24) or Lys11 in the agonist [Ala1,Glu4,Lys11,Arg26,(Met30 or Nle30)]PYY (SEQ ID No:25).

38. The composition as claimed in claim 34 wherein the GAG-binding motif is Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala coupled through an amide bond formed between the C-terminus of the concatameric GAG-binding motif and the epsilon amino group of Lys18 in [Lys18,Gln34]PP (SEQ ID No: 24) or Lys11 in the agonist [Ala1,Glu4,Lys11,Arg26,(Met30 or Nle30)]PYY (SEQ ID No: 25).

39. The composition as claimed in claim 25 wherein, in the agonist, the GAG binding motif is covalently linked to the C- or N-terminus of the agonist, either directly or via a linker radical.

40. The composition as claimed in claim 39 wherein, in the agonist, the GAG binding motif is covalently linked either directly or via a linker radical to the N-terminus of the agonist.

41. The composition as claimed in claim 39 wherein, in the agonist, the GAG-binding motif comprises the amino acid sequence XBBXBX and/or XBBBXXBX, wherein B is a basic amino acid residue and X is any amino acid residue.

42. The composition as claimed in claim 39 wherein, in the agonist, the GAG-binding motif comprises the amino acid sequence [XBBBXXBX]ₙ where n is 1 to 5, B is a basic amino acid residue and X is any amino acid residue.

43. The composition as claimed in claim 39 wherein the peptide is Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala[Gln34]PP (SEQ ID NO:26), [Ala1,Glu4,N-(Ala-Arg-Arg-Arg-Ala-Ala-Arg-Ala)₃-Lys13,Arg26,Nle30]PYY (SEQ ID No: 38), or [Cys2,N-{(Ala-Arg-Arg-Arg-Ala-Ala-Arg-Ala)₃-Lys13,D-Cys27,Gln34]PP (SEQ ID No: 32).

44. The composition as claimed in claim 25 wherein, in the agonist. the PEG is a polyethylene glycol or a polyethylene oxide having a molecular weight of at the most about 20 kDa.

45. The composition as claimed in claim 25 wherein the agonist is [Lys11,Gln34]PP PEGylated at Lys11, or [Ala1,Glu4,Lys18,Arg26,(Met30 or Nle30)]PYY PEGylated at Lys18, or [Ala1,Glu4,N-PEG5000-Lys13,Arg26,Nle30]PYY (SEQ ID No: 39), or [Cys2,N-{N′-(21-amino-4,7,10,13,16,19-hexaoxaheneicosanoyl)}-gammaglutamoyl-Lys13,D-Cys27,Gln34]PP (SEQ ID No: 33).

46. The composition as claimed in claim 25 wherein, in the agonist, the helix inducing peptide is covalently linked, either directly or via a linker radical, to the C- or N-terminus of the agonist.

47. The composition as claimed in claim 25 wherein, in the agonist, the helix inducing peptide is covalently linked, either directly or via a linker radical, to the N-terminus of the agonist.

48. The composition as claimed in claim 46 wherein the helix inducing peptide has 4-20 amino acid residues selected from the group consisting of Ala, Leu, Ser, Thr, Tyr, Asn, Gln, Asp, Glu, Lys, Arg, His, Met, Orn, and amino acid residues of formula —NH—C(R1)(R2)-CO— wherein R1 is hydrogen and R2 is optionally substituted C1-C6 alkyl, phenyl or phenylmethyl, or R1 and R2 taken together with the C atom to which they are attached form a cyclopentyl, cyclohexyl or cycloheptyl ring.

49. The composition as claimed in claim 46 wherein the helix inducing peptide comprises 4, 5 or 6 Lys residues.

50. The composition as claimed in claim 46 wherein the agonist is Lys-Lys-Lys-Lys-Lys-Lys-Lys-[Gln34]-PP.

51. The composition as claimed in any of claim 26 wherein, in the agonist, the serum albumin binding motif, or GAG binding motif, or PEG radical is, or forms part of, a side chain of a backbone carbon corresponding to any of the following positions of PYY or PP: 1, 3, 6, 7, 10, 11, 12, 13, 15, 16, 17, 18, 19, 21, 22, 23, 25, 26, 28, 29, 30 and 32, or corresponding to any of the following positions of NPY: 1, 3, 6, 7, 10, 11, 12, 14, 15, 16, 17, 18, 19, 21, 22, 23, 25, 26, 28, 29, 30 and 32.

52. The composition as claimed in claim 51 wherein, the agonist is of type (c), and the serum albumin binding motif or GAG binding motif, or PEG radical is, or forms part of, a side chain of a backbone carbon corresponding to any of the following positions of PYY, NPY or PP: 2, 5, 8, 9, 13, 14, 20, and 24.

53. The composition as claimed in claim 51 wherein the agonist is as claimed in claim 12 and the serum albumin binding motif or GAG binding motif, or PEG radical is, or forms part of, a side chain on the backbone —(CH₂)ₙ-linker radical.

54. A Y receptor agonist other than [Gln34]PP(SEQ ID No:4) or [Ile31,Gln34]PP(SEQ ID No:5), which is selective for the Y2 and Y4 receptors over the Y1 receptor, as defined in claim 1.

55. A Y receptor agonist which is selective for the Y2 and Y4 receptors over the Y1 receptor selected from
[N—(N′-hexadecanoyl)-gammagluatamoyl-Lys13,Gln34]PP (SEQ ID No: 34)
[N-(Ala-Arg-Arg-Arg-Ala-Ala-Arg-Ala)₃-Lys18,Gln34)PP (SEQ ID No: 35)
[N-PEG5000-Lys13,Gln34]PP (SEQ ID No: 36)
[Val31,Gln34]PP (SEQ ID No: 6)
[Leu30,Gln34]PP (SEQ ID No: 7)
[Nle30,Gln34]PP (SEQ ID No: 8)
[Leu26,Gln34]PP (SEQ ID No: 9)
[His26,Gln34]PP (SEQ ID No: 10)
[Ile3,Gln34]PP (SEQ ID No: 11)
[Ala1,Glu4,Arg26,(Met30 or Nle30)]PYY (SEQ ID No: 12)
[Ala1,Glu4,N—(N′-hexadecanoyl)-gammagluatamoyl-Lys13,Arg26,Nle30]PYY (SEQ ID No: 37).
[Ala1,Glu4,N-(Ala-Arg-Arg-Arg-Ala-Ala-Arg-Ala)₃-Lys13,Arg26,Nle30]PYY (SEQ ID No: 38)
[Ala1,Glu4,N-PEG5000-Lys13,Arg26,Nle30]PYY (SEQ ID No: 39)
[Ala1,Glu4,Arg26(Met30 or Nle30)]NPY (SEQ ID No: 13)
[Ala1,GIu4]PYY (SEQ ID No: 14) and [Ala1,Glu4]NPY (SEQ ID No: 15)
[Arg26, (Met30 or Nle30)]PYY (SEQ ID No: 16) and [Arg26, (Met30 or Nle30)]NPY (SEQ ID No: 17)
[Glu4,(Met30 or Nle30)]PYY (SEQ ID No: 18) and (Glu4, (Met30 or Nle30)]NPY (SEQ ID No: 19)
[Glu4,Arg26]PYY (SEQ ID No: 20) and (Glu4,Arg26]NPY (SEQ ID No: 21)
[Cys2,D-Cys27, Gln34]PP (SEQ ID No: 22)
[Cys2, Aoc5-24,D-Cys27,Gln34]PP (SEQ. ID No: 23)
[Cys2.Lys13,D-Cys27,Gln34)PP (SEQ ID No: 30)
[Cys2,N-(8-(8-gammaglutamoylamino-octanoylamino)-octanoyl-Lys13,D-Cys27,Gln34]PP (SEQ ID No: 31)
[Cys2,N—[(Ala-Arg-Arg-Arg-Ala-Ala-Arg-Ala)₃-Lys13,D-Cys27,Gln34]PP (SEQ ID No: 32)
[Cys2,N—(N′-{21-amino-4,7,10,13,16,19-hexaoxahenaicosanoyl))-gammaglutamoyl-Lys13,D-Cys27, Gln34]PP (SEQ ID No: 33).
and conservatively substituted analogues thereof.

56. A Y receptor agonist which is selective for the Y2 and Y4 receptors over the Y1 receptor which is [Ala1,Glu4,Arg26, (Met30 or Nle30)]PYY or a conservatively substituted analogue thereof.

57. A method of treatment of conditions responsive to activation of Y2 and/or Y4 receptors the method comprising administering to a patient in need thereof an effective amount of a Y2 and Y4 selective receptor agonist as defined in claim 1.

58. The method as claimed in claim 57, wherein the condition treated is one for which regulation of energy intake or energy metabolism, control of intestinal secretion, decrease of gastro-intestinal tract motility, or induction of angiogenesis, is indicated.

59. The method as claimed in claim 58 wherein the condition treated is one for which induction of angiogenesis is indicated, and wherein the Y2 and Y4 selective receptor agonist comprises a GAG-binding motif.

60. The method as claimed in claim 58 wherein the condition treated is one for which induction of angiogenesis is indicated, and wherein the Y2 and Y 4 selective receptor agonist comprises a serum-binding motif.

61. The method as claimed in claim 58 wherein the condition treated is one for which induction of angiogenesis is indicated, and wherein the Y2 selective receptor agonist is PEGylated.

62. The method as claimed in claim 58 wherein the treatment is to decrease the rate of gastric emptying.

63. The method as claimed in claim 58, wherein the condition treated is obesity or overweight, a condition in which obesity or overweight is considered a contributory factor.

64. The method as claimed in claim 63 wherein the condition treated is bulimia, bulimia nervosa, Syndrome X (metabolic syndrome), diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis. coronary artery disease, myocardial infarction, peripheral vascular disease, stroke, thromboembolic diseases, hypercholesterolemia. hyperlipidemia, gallbladder disease, osteoarthritis, sleep apnea, reproductive disorders such as polycystic ovarian syndrome, or cancer of the breast, prostate, or colon.

65. A method as claimed in claim 63 wherein the agonist is administered to a patient in the fasted state.

66. The method as claimed in claim 58, wherein the condition treated is diarrhoea or hyper-secretion from intestinal stomia.

67. A method as claimed in, claim 57 wherein the agonist is administered to a patient via a parenteral route.

68. A composition as claimed in claim 1 comprising one or more of the Y2 and Y4 selective receptor agonists and a pharmaceutically acceptable excipient.

69. A composition as claimed in claim 1 comprising one or more of the Y2 and Y4 selective agonists and a cosmetically acceptable excipient.
